(19) United States
(12) Patent Application Publication
(10) Pub. No.: US 2004/0034882 A1
(43) Pub. Date: Feb. 19, 2004

(54) Corticotropin releasing factor receptor 2 deficient mice and uses thereof

(76) Inventors: Wylie W. Vale, La Jolla, CA (US); Tracy L. Bale, Newtown Square, PA (US); Kuo-Fen Lee, Del Mar, CA (US); George W. Smith, Orondaga, MI (US)

Correspondence Address: Benjamin Aaron Adler, ADLER & ASSOCIATES, 8011 Candle Lane, Houston, TX, 77071, US

(21) Appl. No.: 10/442,797

(22) Filed: May 21, 2003

Related U.S. Application Data
(63) Continuation-in-part of application No. 09/714,692, filed on Nov. 16, 2000.
Application No. 09/714,692 is a continuation-in-part of application No. 09/616,937, filed on Jul. 14, 2000, now Pat. No. 6,353,152.
(60) Provisional application No. 60/144,261, filed on Jul. 15, 1999.

(51) Int. Cl. A01K 67/027

(52) U.S. Cl. 800/18

(57) ABSTRACT

The present invention provides transgenic mice deficient in corticotropin releasing factor receptor 2 (CRFR2). Mice deficient for CRFR1 exhibit decreased anxiety-like behavior and a decreased stress response. In contrast, CRFR2 null mutant mice are hypersensitive to stress and display increased anxiety-like behavior. These mice are useful for the study of anxiety, depression, and the physiology of the HPA axis. CRFR2 null mutant mice also exhibit increased angiogenesis in all tissues examined. Thus, CRFR2 antagonists may be used to stimulate angiogenesis for the treatment of various conditions. In contrast, CRFR2 agonists may be used to inhibit angiogenesis. A combination of urocortin and bFGF was observed to stimulate rapid hair growth. The CRFR2 mutant mice are also useful for the study of the effects of CRFR2 deficiency on homeostatic responses to stress, including a high-fat diet, repeated cold stress, and glucose and insulin challenges. The mutant mice to such stresses enable methods to screen compounds for effects on homeostasis, which are useful in screening compounds to provide treatments for pathological conditions related to the regulation of homeostasis, including obesity and type 2 diabetes.

### CROSS-REFERENCE TO RELATED APPLICATION

This non-provisional patent application is a continuation in part of U.S. Ser. No. 09/714,692 filed Nov. 16, 2000, which is a continuation in part of U.S. Ser. No. 09/616,937, now U.S. Pat. No. 6,353,152, filed Jul. 14, 2000 which claims benefit of provisional patent application U.S. Serial No. 60/144,261, filed Jul. 15, 1999, now abandoned.

### FEDERAL FUNDING LEGEND

This invention was produced in part using funds from the Federal government under grant no. NIH DK-26741 and NRSA fellowships DK09841 and DK09551. Accordingly, the Federal government has certain rights in this invention.

### BACKGROUND OF THE INVENTION

1. Field of the Invention

The present invention relates generally to the fields of metabolic regulation, neurobiology, endocrinology, and psychiatry. More specifically, the present invention relates to the study of homeostatic regulatory mechanisms and anxiety and to mice deficient for corticotropin releasing factor receptor 2.

2. Description of the Related Art

Corticotropin releasing factor (CRF) is a critical coordinator of the hypothalamic-pituitary-adrenal (HPA) axis. In response to stress, corticotropin releasing factor released from the paraventricular nucleus of the hypothalamus (PVN) activates corticotropin releasing factor receptors on anterior pituitary corticotropes, resulting in release of adrenocorticotropic hormone (ACTH) into the bloodstream. ACTH in turn activates ACTH receptors in the adrenal cortex to increase synthesis and release of glucocorticoids (1).

The receptors for CRF, CRFR1 and CRFR2 are localized throughout the CNS and periphery. While CRF has a higher affinity for CRFR1 than for CRFR2, urocortin (UCN), a CRF-related peptide, is thought to be the endogenous ligand for CRFR2 since it binds with almost 40-fold higher affinity than does CRF (2). CRFR1 and CRFR2 share approximately 71% amino acid sequence similarity and are distinct in their localization within the brain and peripheral tissues (3-6). CRFR1 is expressed mainly in the pituitary gland, cortex, cerebellum, hindbrain, and olfactory bulb, whereas CRFR2 is found in the lateral septum, ventral medial hypothalamus (VMH), choroid plexus, and many peripheral sites (3, 7).

Mice deficient for CRFR1 have decreased HPA axis hormone levels, an impaired stress response, and decreased anxiety-like behavior (8, 9). These results coincide with those obtained using CRFR1 specific antagonists in vivo (10-12). In contrast, CRFR2 specific antagonists are not currently available, and since its cloning in 1995, little has been elucidated regarding the physiological function of CRFR2. UCN may be the endogenous ligand for CRFR2 and has been shown to be a modulator of feeding when administered centrally (13). Since CRFR2 is localized to the ventral medial hypothalamus, a central site of food intake regulation and satiety, it is possible that urocortin actions on these receptors may affect feeding. Further, peripheral administration of urocortin results in hypotension (2, 14) which may be the result of action at CRFR2 found in vascular endothelial cells (3, 7). Therefore, in order to discern the developmental and physiological roles of CRFR2, CRFR2 null mutant mice were generated and analyzed.

CRF and its family of ligands including UCNI, UCNII, and UCNIII are key regulators of energy balance. This family of neuropeptides has been shown to be important in the regulation of food intake (37-42), anxiety (43-46), and stress (47-52). Although UCNI has a high affinity for both CRFR1 and CRFR2, UCNIII and UCNIII are specific for CRTR2. The roles these receptors play in central nervous system functions have been deciphered through various pharmacological and genetic manipulations. CRFR1 has been shown to be the dominant receptor in activation of the HPA axis in response to stress, as well as a key mediator of anxiety in the limbic system. Intracerebroventricular (icv) infusions of specific antagonists to CRFR1 diminish anxiety-like behaviors and inhibit the HPA axis response to stress. Similarly, mice deficient for CRFR1 have a decreased stress response and display anxiolytic-like behaviors. Results from icv infusion of agonists, antagonists, or antisense oligonucleotides for CRFR2 have been inconsistent (42, 66-69). Although several studies have shown an anxiogenic response of antagonists to CRFR2, others have found little effect or even an anxiolytic response. Mice deficient for CRFR2 display a phenotype in opposition to the phenotype of the CRFR1-deficient mice, with the CRFR2-mutant mice being hypersensitive to stress (70, 71) and displaying anxiogenic-like behaviors (70, 72). Despite the opposing phenotypes produced by single CRFR mutations, mice deficient for both CRFRs display an unexpected phenotype. These mice not only have a more exaggerated impairment of their HPA-stress response than the CRFR1-mutant mice, but they also display sexually dichotomous anxiety-like behaviors (73). Although overall data seem to support a modulatory or inhibitory role for CRFR2 on CRFR1 actions, results from examination of these double-mutant mice bring to light possible independent actions of CRFR2.

Regulation of homeostasis is an important function of the CNS that requires adaptive responses to maintain and support life. CRF has been shown to be a key player in this process because it rapidly mobilizes the organism for behavioral responses to stress. The icv infusion of CRF elevates sympathetic outflow as measured by increased glucose (74, 75), increased brown adipose tissue (BAT) thermogenesis (76), increased uncoupling protein (UCP)-1 in BAT (77), elevated sympathetic nervous activity to BAT (78, 79), increased plasma catecholamines (47, 80), and increased plasma corticosterone (47, 81). Because CRF has a 10-fold higher affinity for CRFR1 than for CRFR2, and the CRF fiber distribution in the CNS more closely matches that of CRFR1, it is likely that these actions of CRF are due to activation of CRFR1 (82). The role CRFR2 plays in energy balance has been less well defined. We have previously shown that mice deficient for CRFR2 have an altered response to the stress of food deprivation such that mutant mice consume less food on refeeding (70). Others have reported significant alterations of CRFR2 expression in the hypothalamus by stress, food deprivation, and leptin (83-86), suggesting a thigh regulation and important role of this receptor in homeostasis. Because CRF and UCNI levels are elevated in the CNS of CRFR2-deficient mice (70, 71), increased activity at CRFR1 is possible and may explain the increased anxiety-like behavior and hypersensitivity to stress in these mice. To examine the role CRFR2 plays in energy balance, we have examined the responses of mice deficient for CRFR2 to perturbations of homeostasis, including repeated cold stress, high-fat diet, and glucose and insulin challenges.

Strong evidence links stress, and the sensitivity of the individual to stressful encounters, to the development of depression. The stress response is essential for adaptation, maintenance of homeostasis, and survival. Chronic stress, however, can accelerate disease processes, cause neural degeneration, and lead to depression or other mood disorders (116). A key factor in the response to stress is the neuropeptide CRF (125). A large body of evidence now ties CRF to the development of depression (114, 115, 93, 107, 120). Clinical studies have found increased CRF and decreased CRF receptors in postmortem examination of suicide victims. Further, excessive activation of the HPA axis has been reported in over half of patients with depression, and these symptoms have been corrected during antidepressant treatment (107). While CRF stimulates the HPA axis in response to stress and plays a key role in activation of anxiety behaviors via activation of CRFR1, other CRF family members such as UCNI, UCNII, and UCNIII may act to decelerate the stress response via activation of CRFR2 (95, 97, 94, 124). Localization of CRF receptors within brain regions thought to be involved in the neural circuitry of depression also supports an involvement of CRF pathways in the pathogenesis of this disease (96). It is clear that the delicate balance of the CRF system is critical for maintenance of mental and physical soundness.

The increased susceptibility of females to depression has been well documented while the underlying mechanisms remain insufficiently studied and virtually unknown. Differences in neuroendocrine pathways or sexually dimorphic brain regions may be key factors influencing sensitivity. The CRF system and stress have not been well examined in relation to gender differences in the development of depression, but may be key factors influencing increased female susceptibility (130). CRFR2-deficient mice have previously been reported to display a hypersensitive HPA axis, anxiogenic-like behavior, and elevated levels of CRF and UCNI (95). As a possible mouse model of depression, male and female CRFR2-deficient mice were examined for depression-like behaviors. In order to determine the possible involvement of CRFR1 activation in the absence of CRFR2, the non-peptide CRFR1 antagonist, antalarmin, was administered prior to testing. Previous studies have demonstrated an antidepressant action of antalarmin in the forced swim test (103). Our studies reveal an involvement of CRF receptors in the development of depression and distinct behavioral sex differences in response to CRFR1 antagonist treatment.

The prior art is deficient in the lack of screening methods for potentially therapeutic compounds and therapeutic applications involving mice deficient for corticotropin releasing factor receptor 2. The present invention fulfills this longstanding need and desire in the art.

### SUMMARY OF THE INVENTION

CRFR2 deficient mice exhibit increased anxiety-like behavior and a hypersensitive HPA axis in response to stress. CRFR1 and CRFR2 null mutant mice provide valuable models of anxiety and depression and may further help delineate the molecular mechanisms underlying these diseases. Study of the corticotropin releasing factor signaling pathway and its role in the management of anxiety and depression may provide the necessary clues required for the effective treatment of these diseases.

Thus, the present invention is directed to a non-natural transgenic mouse with a disruption in at least one allele of the corticotropin releasing factor receptor 2 (CRFR2) such that said mouse does not express corticotropin releasing factor receptor 2 protein from said allele. Preferably, the DNA sequences for exons 10, 11, and 12 of said corticotropin releasing factor receptor 2 allele have been deleted. The transgenic mouse may have these DNA sequences replaced with a neomycin resistance gene cassette. The transgenic mouse may be either heterozygous or homozygous for this replacement. Also included in an embodiment of the present invention are the progeny of a mating between a mouse of the present invention and a mouse of another strain.

Another embodiment of the present invention is the application of a CRFR2 deficient mouse to the study of anxiety or depression and to test the effects of various compounds on anxiety or depression. For example, a method is provided of screening a compound for anxiety modulating activity, comprising the steps of: a) administering said compound to the transgenic mouse of the present invention; b) testing said mouse for anxiety-related behavior; and c) comparing anxiety-like behavior of said mouse with anxiety-like behavior in a second transgenic mouse of the present invention to which said compound was not administered. In addition, a method of screening a compound for depression-modulating activity is provided, comprising the steps of: a) administering said compound to the transgenic mouse of the present invention; b) testing said mouse for depression-like behavior; and c) comparing depression-like behavior of said mouse with depression-like behavior in a second transgenic mouse of the present invention to which said compound was not administered.

Yet another embodiment involves the use of a CRFR2deficient mouse in a similar procedure to screen for compounds that affect blood pressure or angiogenesis.

A further embodiment of the current invention is the application of the CRFR2 deficient mice to the study of the physiology of the HPA axis, e.g., a method of screening a compound for effects on the response of the hypothalamic-pituitary-adrenal axis to stress, comprising the steps of: a) administering said compound to a transgenic mouse of the present invention; b) placing said mouse in a stress-inducing situation; c) monitoring plasma levels of corticosterone and adrenocorticotropic hormone in said mouse; and d) comparing said levels to those in a transgenic mouse of the present invention not placed in said stress-inducing situation.

In yet another embodiment of the current invention, the mice can be used to study the effects of a compound on the response of the HPA axis to stress by monitoring plasma levels of corticosterone and ACTH.

Yet another embodiment of the current invention relates to the use of the mice in the study the effect of corticotropin releasing factor receptor 2 on other proteins such as corticotropin releasing factor and urocortin.

Yet another embodiment of the current invention relates to the use of the mice in the study the effect of corticotropin releasing factor receptor 2 on other proteins such as corticotropin releasing factor and urocortin.

A further embodiment of the current invention is the use of the CRFR2 deficient mice to examine CRFR1 responses unhindered by the presence of CRFR2.

Examination of the CRFR2 null mutant mice reveals that the loss of the CRFR2 gene results in increased vascularization in all tissues examined. Thus, another embodiment of the instant invention is the application of the CRFR2 null mutant mice to the study of molecular mechanisms of angiogenic regulation.

In another embodiment of the instant invention, angiogenesis may be stimulated in a target tissue by administering a CRFR2 antagonist to the tissue. One example of such an antagonist is an antisense nucleotide directed against the CRFR2 gene. Heart, brain, pituitary, gonad, kidney, adipose, and gastrointestinal tract are among the tissues in which such a response may be attained. This stimulation of angiogenesis may prove useful in treating infarctions, strokes, and injuries.

In yet another embodiment of the instant invention, angiogenesis may be inhibited in a target tissue by administering a CRFR2 agonist such as urocortin or CRF. CRFR2 agonist-induced inhibition of angiogenesis may be used in the treatment of cancer and diabetic retinopathy.

A further embodiment of the instant invention is directed to a method of stimulating hair growth by implanting urocortin and bFGF under the area of skin on which hair growth is desired or or contacting urocortin with the skin in a topical composition.

Yet another embodiment of the invention includes a method of screening a compound for effects on a response to stress on homeostasis, comprising the steps of administering said compound to a first wild-type mouse, placing said first wild-type mouse, a second wild-type mouse, and the transgenic mouse of the instant invention in a stress-inducing situation, monitoring said response to stress in said first wild-type and said transgenic mouse; and comparing the response to stress in the wild-type mouse to the response in the transgenic mouse to the response of a second wild-type mouse to which said compound was not administered.

An additional embodiment includes method of treating a pathological condition, comprising the step of administering an effective dose of a compound to an individual in need of such treatment.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1E show the procedure used for the generation of CRFR2-Deficient Mice.

FIG. 1A: Genomic organization of the CRFR2 gene showing the deletion of exons 10, 11, and 12 which code for half of transmembrane domain five through the end of transmembrane domain seven. The targeting construct utilized for homologous recombination is also shown

FIG. 1B: The disrupted allele was detected by Southern Blot analysis of tail DNA isolated from wild type (+/+), heterozygote (±), and null mutant (−/−) mice.

FIG. 1C: Autoradiographic binding of ¹²⁵I-Sauvagine in CRFR2 control (top) and mutant (bottom) mice. Note, no CRFR2 binding in the lateral septum of CRFR2 null mutant mice, while the CRFR1 cortical binding is similar to that of the control mouse.

FIG. 1D: Hematoxylin and eosin (H&E) staining of the adrenal gland. Note no difference in adrenal gland size (upper panels) at 10× magnification or structure (lower panels) at 20× magnification, C, cortex; M, medulla; ZG, zona glomerulosa; ZF, zona fasciculata; ZR, zona reticularis; n=8.

FIG. 1E: H&E staining of the pituitary glands which were mounted on liver for tissue sectioning (upper panels) at 4× magnification, n=8. Pituitary corticotropes were identified with anti-ACTH antibodies (20) (lower panels) at 10× magnification, n=5. P, posterior lobe; I, intermediate lobe; A, anterior lobe. No gross anatomical differences were observed for the pituitary gland or for the corticotrope localization or expression levels of ACTH.

FIGS. 2A-2D show the hypersensitivity of HPA axis to stress in mutant animals. *=significantly different from wild type controls at same time point, p<0.01 by Scheffe post-hoc test. Plasma obtained by unanesthetized retro-orbital eyebleeds.

FIG. 2A: Pre-stress ACTH plasma levels at 7:00 AM, n=16.

FIG. 2B: Basal corticosterone plasma levels for 7:00 AM and 5:00 PM, n=7.

FIG. 2C: Time course of restraint stress effects on ACTH.

FIG. 2D: Corticosterone plasma levels (7:00 AM) are significantly different from wild type control at same time point, n=7.

FIGS. 3A-3B show the effect of 24 hours of food deprivation on food intake in wild type and mutant littermate mice.

FIG. 3A: Food consumption of mutant mice (n=7) basal and following a 24 hr food deprivation period as compared to wild type litter mates (n=10), p<0.001 by Scheffe post-hoc test.

FIG. 3B: Weight of wild type and mutant mice, basal (open bars) and following 24 hrs of refeeding (black bars) following the food deprivation period. Note that there are no differences between groups in basal or refed body weights.

FIGS. 4A-4D demonstrate the increased anxiety-like behavior of mutant animals in the elevated plus maze, (control n=7, mutant n=7; mean±SEM).

FIG. 4A: Percentage of time spent in the open arms (**, p<0.005) and number of visits to the open arms (*, p<0.02) were significantly less for the mutant mice than for the wild type controls.

FIG. 4B: Locomotor activity was not different between control and mutant animals as measured by total closed arm entries (p=0.64) and total arm entries (p=0.38).

FIG. 4C: No differences were found in anxiety-like behavior measured in the light/dark box experiment for time spent in the light portion of the box.

FIG. 4D: No differences were found in anxiety-like behavior measured in the light/dark box experiment for the number of transitions between the light and dark portions.

FIGS. 5A-5E show the increased levels of urocortin and CRF mRNA in the mutant brains. For 4B to 4E, n=3, ±SEM, *, p<0.05; ** p<0.01; ***, p<0.005.

FIG. 5A: Silver grains resulting from in situ hybridization (23) for urocortin mRNA in the rostral EW (upper) at 20× magnification and CRF mRNA in cAmyg (middle) and paraventricular nucleus (lower) at 10× magnification.

FIG. 5B: Semi-quantitative analysis of silver grains was used to determine cell numbers expressing urocortin mRNA in the rostral EW.

FIG. 5C: Average optical density of urocortin mRNA per cell.

FIG. 5D: Optical density of CRF mRNA in the cAmyg.

FIG. 5E: Optical density of CRF mRNA in the paraventricular nucleus.

FIG. 6 shows cardiovascular responses to intravenous infusion of 1.0 μg urocortin in wild type (n=5) and mutant mice (n=3). Note the remarkable muted response of mutant mice to the urocortin injection. *** p<0.005.

FIGS. 7A-7F show tissues from adult CRFR2 control (FIGS. 7A, 7C and 7E) and CRFR2 null mutant (FIGS. 7B, 7D and 7F) mice immunostained with anti-PECAM antibodies. These studies showed an increase in vessel number and size in the anterior pituitary (FIG. 7B), white adipose tissue (FIG. 7D) and dorsal brain surface (FIG. 7F) of CRFR2 null mutant mice as compared to the anterior pituitary (FIG. 7A), white adipose tissue (FIG. 7C) and dorsal brain surface (FIG. 7E) of control mice.

FIGS. 8A-8B show immunostained tissues from embryonic day 11 CRFR2 null mutant and control mice. FIG. 8A shows tissues from the head of CRFR2 null mutant (right) and control (left) mice. FIG. 8B shows tissues from the front paws of CRFR2 null mutant (right) and control (left) mice. No difference in vessel number and size was observed in either the head or front paws.

FIGS. 9A-9C show microfil perfused tissues from adult CRFR2 null mutant (right, FIG. 9A, FIG. 9B and FIG. 9C) and control mice (left, FIG. 9A, FIG. 9B and FIG. 9C). CRFR2 null mutant mice show increased vessel number in the dorsal brain surface (FIG. 9A), large intestine (FIG. 9B) and heart (FIG. 9C).

FIGS. 10A-10F show microfil perfused tissues from adult CRFR2 null mutant (FIG. 10B, FIG. 10D and FIG. 10F) and control mice (FIG. 10A, FIG. 10C and FIG. 10E). The arrows indicate the primary arteries for the kidney (FIGS. 10A and 10B), adrenal gland (FIGS. 10C and 10D) and testis (FIGS. 10E and 10F).

FIGS. 11A-11D show microfil perfused tissues from 3 week old CRFR2 null mutant (FIG. 11B and FIG. 11D) and control mice (FIG. 11A and FIG. 11C). Mutant mice exhibit an increase in the number of blood vessels in the small intestine (FIGS. 11B vs. 11A) and stomach (FIGS. 11D vs. 11C).

FIG. 12 shows a western blot demonstrating increased VEGF expression in white (WAT) and brown (BAT) adipose tissue from CRFR2 null mutant mice.

FIG. 13 shows that surgical implantation of a gel foam sponge impregnated with urocortin and bFGF stimulated hair growth in the area directly over the sponge implant. The mouse on the right received a sponge containing bFGF only. The mouse on the left was implanted with a sponge impregnated with both urocortin and bFGF.

FIGS. 14(A-G) show the metabolic effects of a high-fat diet on wild type and CRFR2-mutant mice. FIG. 14A, start and end weights for CRFR2-mutant (mut) and wild-type (wt) male mice on low- (LF) and high-fat (HF) diets. Mice show similar body weights before and after low- and high-fat diet for 16 wk (n=7). FIG. 14B, total food intake for CRFR2-mutant and wild-type mice during 16 wk on high-fat diet. Mutant mice consumed significantly more high-fat food than wild-type mice did, despite similar body weights (**, P<0.01). FIG. 14C, percentage body fat for mutant and wild-type mice on low- and high-fat diets. CRFR2-mutant mice have significantly lower body fat than wild-type mice on high-fat diet (*L*, P<0.01). No differences were detected for mice on low-fat diet. FIG. 14D, body composition of mutant and wild-type mice was analyzed following 16 wk on high- or low-fat diet. Percentage body water (H2O), ash, and FFDM for mutant and wild-type mice on high-fat diet showing increased components for mutant mice, compared with littermates (**, P<0.01). Plasma lipid levels for CRFR2-mutant and wild-type mice on high-fat diet were also determined at the end of the 16-wk study. FIG. 14E, plasma triglyceride (trigly) and cholesterol (chol) levels showing decreased levels for mutant mice (***, P<0.001). FIG. 14F, decreased free fatty acid levels for mutant mice (L***, P<0.001). FIG. 14G, feed efficiency for mutant and wild-type mice on low-or high-fat diet. Feed efficiency is calculated as gram weight gained per gram food consumed. CRFR2-mutant mice have a lower feed efficiency than wild-type mice following 16 wk on the high-fat diet (*, P<0.05). All data are displayed as the mean ±SEM.

FIGS. 15(A-F) show the metabolic effects of cold stress. FIG. 15A, body weight of CRFR2-mutant (mut) and wild-type (wt) mice during the daily cold stress (*, P<0.05, n=10). Mutant mice lose weight during the cold stress, Whereas wild-type mice maintain their body weight. FIG. 15B, food intake for mutant and wild-type mice during the cold stress (*, P<0.05). Initially, mutant mice eat less than wild-type mice. However, although their food intake is similar after the first week, the mutant mice still weigh less. FIG. 15C, feed efficiency for CRFR2-mutant and wild-type mice during the cold stress (*, P<0.01). Feed efficiency is measured as the gram of weight gained per gram of food consumed. Body composition of mice following repeated acute cold stress. FIG. 15D, percentage body fat of mutant and wildtype mice showing decreased body fat of mutant mice despite similar body weights (***, P<0.001). Mutant mice have slightly increased water, ash, and FFDM, compared with wild-type mcie (***, P<0.001; **, P<0.005). Plasma lipids following the cold stress show no significant differences between genotypes for cholesterol or triglycerides (FIG. 15E) or free fatty acids (FIG. 15F). All data are displayed as the mean ±SEM.

FIGS. 16(A-D) show glucose and insulin responses. Glucose challenge glucose levels for male (FIG. 16A) (n=10), CRFR2-mutant (mut) and wild-type (wt) mice. Mutant mouse glucose levels do not rise as high as wild-type levels following glucase challenge and decline at a faster rate (*, P<0.05; **, P<0.01). FIG. 16B, insulin tolerance test in male (n=20) CRFR2-mutant and wild-type mice. Mutant mouse glucose levels decrease faster than wild-type levels following insulin administration (*, P<0.05). Glucose (FIG. 16C) and insulin (FIG. 16D) levels of male CRFR2-mutant and wild-type mice before and following 4 wk of high-fat diet (n=12). Wild-type glucose levels significantly rise during the 4 wk of high-fat diet, whereas mutant levels remain unchanged (high-fat baseline significantly different between wild-type and mutant (*, P<0.05). Insulin levels also rise to a greater extent in the wild-type mice (high-fat baseline significantly different from regular diet baseline (**, P<0.01). All data are displayed as the mean ±SEM.

FIGS. 17(A-C) show adipose cell size and UCP1 expression. Representative histology of white adipose tissue (WAT) and brown adipose tissue (BAT) is shown in FIGS. 17A and 17B, respectively, showing that mutant mice have smaller adipocytes, compared with wild-type mice. Cell counts using number of nuclei per area indicates more cells in BAT from mutant mice (175±14) than wild-type mice (105±9), suggesting a smaller cell size. FIG. 17C, changes in protein levels for UCP1 in CRFR2-mutant and wild-type BAT (40 μg protein per lane).

FIGS. 18(A-B) show locomotor activity for CRFR2mutant and wild-type mice. FIG. 18A, 24-h horizontal activity counts for CRFR2-mutant and wild-type male mice (n=4). FIG. 18B, twenty-four-hour rearing behavior for CRFR2-mutant and wild-type male mice. Data are displayed as the mean ±SEM. The black bar represents the dark cycle.

FIGS. 19(A-C) show measurement of depression-like behaviors in a forced swim test. FIG. 19A, both male and female mutant mice showed increased immobile time during 5 min forced swim compared to wild-type mice (n=12). CRFR2-deficient females showed significantly greater immobile time compared to wild type females (***, P<0.001). CRFR2-deficient males also showed significantly increased immobility compared to wild type males (**, P<0.01). Overall, females showed significantly greater immobile time compared to their respective males of the same genotype (***, P<0.001 compared to male mutant; *, P<0.05). FIG. 19B, both male and female mutant mice demonstrated decreased swim time in the forced swim test compared to wild-type littermates (n=12) (*, P<0.05; **, P<0.01). FIG. 19C, female mice deficient for CRFR2 spent significantly less time climbing during the forced swim test compared to female wild type littermates (***, P<0.001). No significant differences were detected between male mutant and wild type mice for climbing time (n=12).

FIGS. 20(A-C) show forced swim test results following pre-treatment with antalarmin in female CRFR2-deficient mice. FIG. 20A, female mutant mice treated with antalarmin (7.5 mg/kg) one hour prior to testing showed decreased immobile time compared to vehicle treated mutant mice (n=12) (***, P<0.001). This effect remained evident 24 (n=6) and 72 (n=6) hours following treatment (***, P<0.001). Ξ, basal wild type female immobile levels for comparison. FIG. 20B, female mutant mice treated with antalarmin displayed increased swim time compared to vehicle treated mutant females 1 (n=12, 24 (n=6), and 72 (n=6) hours following treatment (*, P<0.05; ***, P<0.001). Ξ, basal wild type female swim levels for comparison. FIG. 20C, female mutant mice treated with antalarmin showed increased climbing time 24 (n=6) and 72 (n=6) hours after treatment compared to vehicle treated females (*, P<0.05). No difference was detected in climbing time 1 hour after treatment (n=12). Ξ, basal wild type female climbing levels for comparison.

FIGS. 21(A-C) show forced swim test results following pre-treatment with antalarmin in male CRFR2-deficient mice. FIG. 21A, male mutant mice treated with antalarmin (7.5 mg/kg) one hour prior to testing showed decreased immobile time compared to vehicle treated mutant mice (n=12) (***, P<0.001). This effect remained evident 24 hours (n=6) following treatment (**, P<0.01). No difference in immobile time was detected 72 hours (n=6) following treatment. Ξ, basal wild type male immobile levels for comparison. FIG. 21B, male mutant mice treated with antalarmin one hour prior to testing showed increased swim time compared to behicle treated mutant mice (n=12; ***, P<0.001). This effect remained evident 24 hours (n=6) following treatment (*, P<0.05). No difference in swim time was detected 72 hours (n=6) after treatment. Ξ, basal wild type male swim levels for comparison. FIG. 21C, male mutant mice treated with antalarmin showed increased climbing time 24 hours (n=6) after treatment compared to vehicle treated males (*, P<0.05). No difference was detected between antalarmin and vehicle treated males in climbing time 1 hour (n=12) or 72 hours (n=6) after treatment. Ξ, basal wild type male climbing levels for comparison.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins Eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

As used herein, the term “cDNA” shall refer to the DNA copy of the mRNA transcript of a gene.

As used herein the term “screening a library” shall refer to the process of using a labeled probe to check whether, under the appropriate conditions, there is a sequence complementary to the probe present in a particular DNA library. In addition, “screening a library” could be performed by PCR.

As used herein, the term “PCR” refers to the polymerase chain reaction that is the subject of U.S. Pat. Nos. 4,683,195 and 4,683,202 to Mullis, as well as other improvements now known in the art.

The amino acids described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J Biol. Chem., 243:3552-59 (1969), abbreviations for amino acid residues are known in the art.

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. Eukaryotic promoters often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included near the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide”, as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementary with the sequence or hybridize therewith and thereby form the template for the synthesis of the extension product.

As used herein, the terms “restriction endonucleases” and “restriction enzymes” refer to enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or ancestor by mitosis. A “cell line” is a clone of a primary cell that is capable of stable growth in vitro for many generations.

In general, expression vectors containing promoter sequences which facilitate the efficient transcription of the inserted DNA fragment are used in connection with the host. The expression vector typically contains an origin of replication, promoter(s), terminator(s), as well as specific genes which are capable of providing phenotypic selection in transformed cells. The transformed hosts can be fermented and cultured according to means known in the art to achieve optimal cell growth.

Methods which are well known to those skilled in the art can be used to construct expression vectors containing appropriate transcriptional and translational control signals. See for example, the techniques described in Sambrook et al., 1989, Molecular Cloning: A Laboratory Manual (2nd Ed.), Cold Spring Harbor Press, N.Y. A gene and its transcription control sequences are defined as being “operably linked” if the transcription control sequences effectively control the transcription of the gene. Vectors of the invention include, but are not limited to, plasmid vectors and viral vectors.

The current invention is directed to mice deficient in CRFR2, which were generated to discern the developmental and physiological roles of CRFR2 in anxiety and HPA axis circuitry. This has been done by deleting exons 10, 11, and 12 of corticotropin releasing factor receptor 2. In the present invention, these sequences have been replaced with a neomycin resistance gene cassette. The mice may be either heterozygous of homozygous for the CRFR2 deficiency and may be crossed with mice of another strain.

The present invention is also directed to the application of the CRFR2 deficient mice in the study of anxiety and depression, including methods of testing a compound for anxiety or depression modulating activity, including a CRFR1 antagonist. One possible test for depression-like behavior is a forced swim test. Depression-like behavior may be compared between male and female mice, in order to determine the effects of differences in males and females to the susceptibility to depression. A possible embodiment of the invention includes a method of treating a pathological condition related to depression, comprising the step of administering an effective dose of a compound exhibiting depression-modulating effects to an individual in need of such treatment. Compounds which affect blood pressure and angiogenesis can also be screened using the CRFR2 mice.

The current invention is also directed to use of the CRFR2 deficient mice in the study of the molecular physiology of the hypothalamic-pituitary-adrenal (HPA) axis. The mice can be used to test the effects of a compound on the response of the HPA axis to stress.

The current invention is also directed to the use of the transgenic mice to study the molecular functions of corticotropin releasing factor receptor 2 on corticotropin releasing factor, corticotropin releasing factor receptor 1, urocortin, and other CRF and urocortin receptors.

In addition, the present invention can be used to study the responses and activities of CRFR1 in a CRFR2 negative environment. In this manner, CRFR1 responses can be studied unhindered by CRFR2 modulation.

The instant invention is also directed to the use of the CRFR2 null mutant mice to the molecular regulation of angiogenesis.

The instant invention is also directed to a method of stimulating increased angiogenesis by administering a CRFR2 antagonist to a target tissue. One manner in which this may achieved is through the use of an antisense nucleotide directed against CRFR2. Heart, brain, pituitary, gonad, kidney, adipose, and gastrointestinal tract are among the tissues in which such a response may be attained. The instant invention will prove useful in stimulating increased angiogenesis following infarction, stroke, and injury.

The instant invention is also directed to a method of inhibiting angiogenesis by administering a CRFR2 agonist to a target tissue such as heart, brain, pituitary, gonad, kidney, adipose, or gastrointestinal tract tissues. CRFR2 agonists include urocortin and CRF. Cancer and diabetic retinopathy are examples of conditions which may be responsive to a CRFR2 agonist induced inhibition of angiogenesis.

The instant invention is directed to a method of stimulating hair growth comprising the step: contacting urocortin with a region of skin on which hair growth is desired. In one aspect, the urocortin may be implanted under the skin. Although urocortin may be useful alone, bFGF may also be administered to the skin before urocortin, after urocortin or simultaneously with urocortin. Urocortin may also be contained in a composition with bFGF.

The instant invention is also drawn to a method of screening a compound for effects on a response to stress on homeostasis, comprising the steps of administering said compound to a first wild-type mouse, placing said first wild-type mouse, a second wild-type mouse, and the transgenic mouse of the instant invention in a stress-inducing situation, monitoring said response to stress in said first wild-type and said transgenic mouse; and comparing the response to stress in the wild-type mouse to the response in the transgenic mouse to the response of a second wild-type mouse to which said compound was not administered. The stress-inducing situation may be a high-fat diet, repeated cold stress, glucose challenge, and insulin challenge. The monitoring of said response may include the analysis of body composition, plasma lipid analysis, tissue histology, Western blot analysis, and analysis of locomotor activity. The responses of the first wild type mouse and the transgenic mouse to said high-fat diet may consist of lower body fat but higher food intake, no elevation in plasma glucose levels, and a slight rise in plasma insulin levels compared with the second wild type mouse to which the compound was not administered. The response of the first wild type mouse and the transgenic mouse to repeated cold stress may include weight loss, lower feed efficiency, and lower body fat compared to the second wild type mouse. The response of the first wild type mouse and the transgenic mouse to glucose challenge may comprise a lower peak plasma glucose level compared to the second wild type mouse. The response of the first wild type mouse and the transgenic mouse to insulin challenge may include a lower peak plasma glucose level, a more rapid decline in plasma glucose levels, and an increase in insulin sensitivity compared to the second wild type mouse. The compound may be an antagonist of CRFR2 activity or an agonist of CRFR1 activity, because either of these compounds may be expected to produce effects similar to the CRFR2 deficiency in the mutant mice.

Another embodiment of the present invention provides a method of treating a pathological condition, comprising the step of administering an effective dose of a compound to an individual in need of such treatment, wherein said pathological condition may include obesity and type 2 diabetes.

The following examples are given for the purpose of illustrating various embodiments of the invention and are not meant to limit the present invention in any fashion.

### EXAMPLE 1

Generation of the CRFR2 Deficient Mice

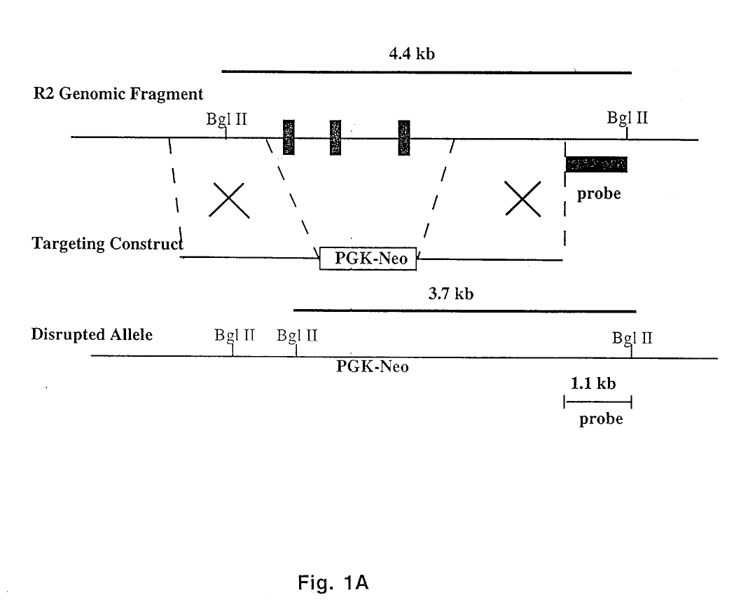

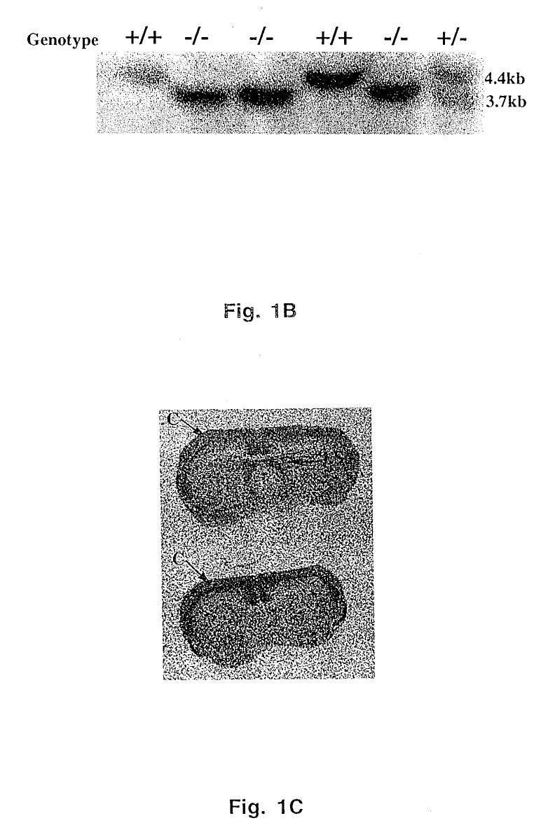

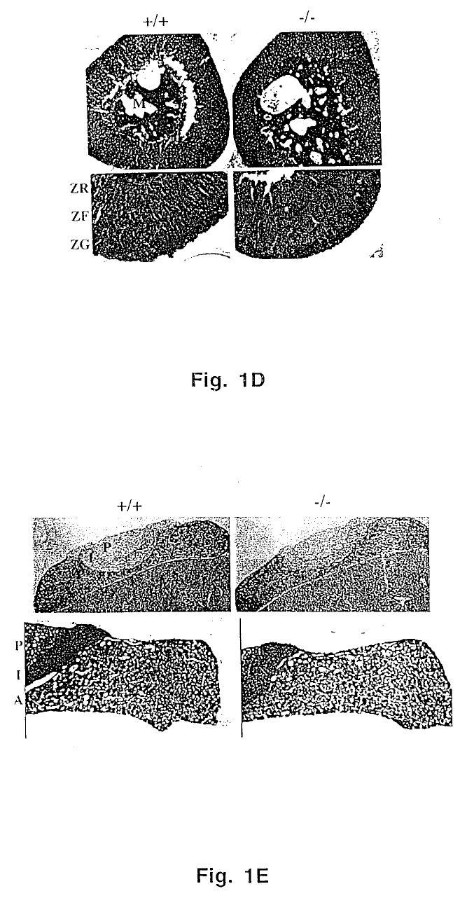

For the construction of CRFR2 null mutant mice, a genomic clone DNA containing the CRFR2 locus was isolated from a mouse strain 129 genomic DNA library. From this clone, a targeting vector was constructed in which the exons 10, 11, and 12 of the CRFR2 gene encoding the beginning of the fifth transmembrane domain through the end of the seventh transmembrane domain were replaced with a neomycin resistance gene cassette (FIG. 1A). The resulting plasmid DNA was linearized with Not I and electroporated in to J1 embryonic stem (ES) cells as previously described (8). After selection in 0.2 mg/ml G418 (active form) for 7-9 days, neomycin resistant clones were individually selected and screened for the presence of the disrupted CRFR2 allele by Southern blot analysis.

Positive ES clones were injected into C57 BL/6 blastocysts to generate chimeric mice. Chimeric males were crossed to C57BL/6 females and germ-line transmission of the disrupted allele was determined by Southern analysis of tail DNA collected from F1 pups displaying agouti coat color (FIG. 1B).

### EXAMPLE 2

Analysis of CRFR1 and CRFR2 expression in CRFR2 Deficient Mice

To determine if the targeted deletion resulted in a null mutation of the CRFR2 gene, receptor autoradiography was performed on brain sections from wild type control and mutant animals.

Slides containing 20 μm sectioned brain tissue were thawed at room temperature and washed twice for 10 min. in 50 mM Tris buffer (pH 7.4) at room temperature. Sections were then incubated in buffer containing 50 mM Tris (pH 7.4), ¹²⁵I-Sauvagine, 10 mM MgCl₂, 0.1% BSA, and 0.05% bacitracin for 60 min. at room temperature. Nonspecific binding was defined in adjacent sections that were exposed to both ¹²⁵I-Sauvagine and 1 μm cold sauvagine. After the incubation period, slides were washed in a 50 mM Tris buffer plus 0.01% Triton X-100 at 4 C. twice for 5 min. each. Slides were rapidly dipped in deionized water, dried and apposed to film for 3 days.

In the mutant mice, no binding in brain regions specific to CRFR2 (lateral septum) was detected, yet binding to CRFR1 in the cortex was retained (FIG. 1C). These results demonstrate that the disruption of the CRFR2 gene resulted in a null mutation in these mice. Mutant mice were fertile and transmitted the mutant allele in a Mendelian fashion.

### EXAMPLE 3

Histological analysis of CRFR2 Deficient Mice

To determine whether the development of the HPA axis was compromised in the CRFR2 deficient mice, the pituitary and adrenal glands of male mice 10-12 week of age were sectioned and stained with hematoxylin and eosin (H&E). Briefly, mice were perfused with 4% paraformaldehyde (PFA). Tissues were removed, postfixed overnight at 4 C., and cryoprotected in 30% sucrose in PBS. Tissues were sectioned at 12 μm thickness and stained with hematoxylin and eosin. The results showed no obvious differences in structure or cell types (FIGS. 1D-1E).

In addition, pituitary sections were stained with anti-ACTH antibodies. The pituitaries were sectioned, postfixed in 4% PFA for 5 min., rinsed in PBS, and stained with ACTH antibody as described previously (6). No qualitative differences were noted between wild type and mutant corticotropes (FIG. 1E).

### EXAMPLE 4

Corticosterone and ACTH Levels in CRFR2 Deficient Mice

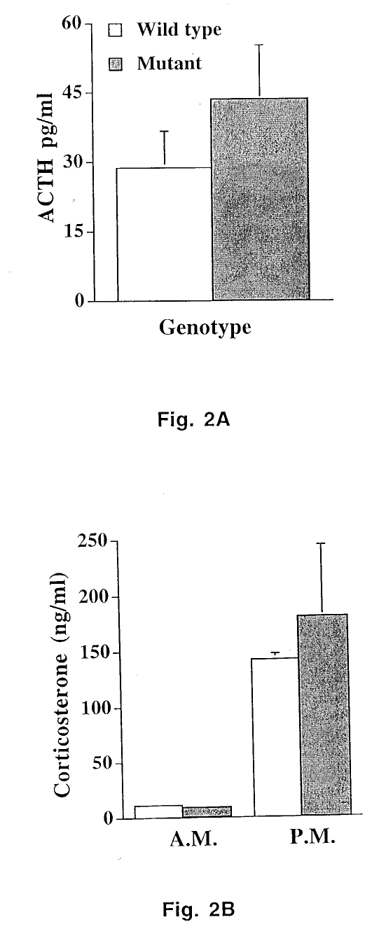

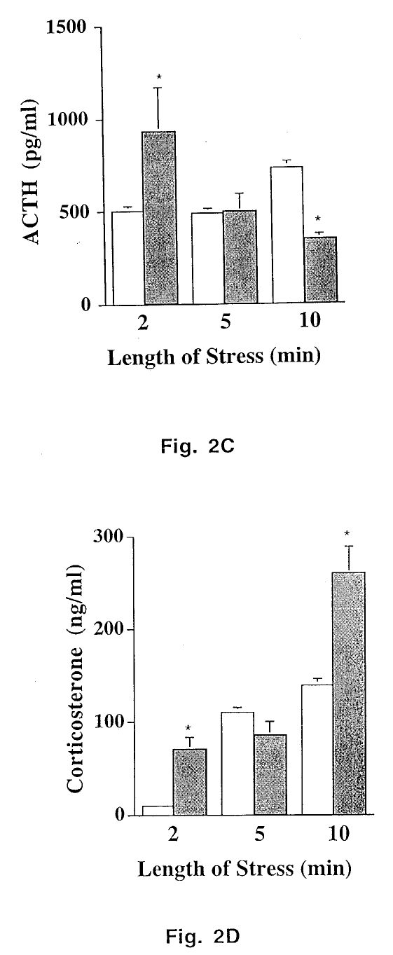

For corticosterone and ACTH analyses, plasma was obtained from individually housed male mice of 10-12 weeks of age. Samples were collected by retro-orbital eye bleed from unanesthetized animals within 30 sec of disturbance of the cage. Basal AM samples were collected at 7:00 AM. Basal PM samples were collected at 5:00 PM. Corticosterone assay (ICN Biomedicals, Dosta Mesa, Calif.) used 5 μl plasma and the ACTH assay (Nichols Institute Diagnostics, San Juan Capistrano, Calif.) used 50 μl plasma as measured in duplicate by radioimmune assay kits. Normal basal levels of ACTH and corticosterone were found in the mutant and control animals (FIGS. 2A-2B), consistent with the finding that ACTH levels are unaffected in the brain.

### EXAMPLE 5

Effects of Stress on the HPA Axis Response in CRFR2 Deficient Mice

In order to examine the HPA axis response to stress, animals were subjected to physical restraint-stress for increasing lengths of time. Blood samples were collected immediately following either 2, 5, or 10 min. of restraint stress in a 50 ml conical tube (plastic conical tube with the bottom removed). Plasma samples were immediately centrifuged and stored at −20C. until the assay was conducted.

ACTH levels in the mutant animals were significantly elevated and peaked following only two minutes of restraint stress (FIG. 2C). In contrast, ACTH levels in control animals peaked following ten minutes of restraint. Similarly, corticosterone levels in the mutant animals were significantly elevated following two minutes of restraint, whereas control animal levels increased following five minutes of the stress (FIG. 2D). These results demonstrated a hypersensitive response of the HPA axis to stress in the mutant mice.

### EXAMPLE 6

CRFR2 Deficient Mice are Sensitive to Food Deprivation

Since CRFR2 is abundant in the VMH and since previous studies had shown an anorectic effect of icv urocortin (13), basal feeding and weight gain were measured in the mutant and wild type litter mates.

Basal feeding was measured in individually housed 12-16 week old male litter mates. Mice and their food pellet were weighed daily at 09:00 hrs. For the food deprivation experiment, control and mutant litter mates were individually housed and their basal food intake and weight was established. Mice were food deprived for 24 hrs beginning at 12:00 hrs, but had water ad libidum. Following the food deprivation period, mice were weighed and given a pre-weighed food pellet. Food pellets were then weighed every two hours until lights off (18:00 hrs). Food pellets and mice were again weighed the following morning. Weight loss during the food deprivation as well as total food consumption and weight gain over the 24 hr period following the food deprivation were recorded.

Basal feeding and weight gain in CRFR2 null mutant (mut) male mice were similar to that of wild type (wt) litter mates (24 hr basal food consumption wt=4.3±0.24 g, mut=4.6±0.23 g; body weight wt=21.7±0.66 g, mut=21.2±0.50 g; n=10, averages are ±sem).

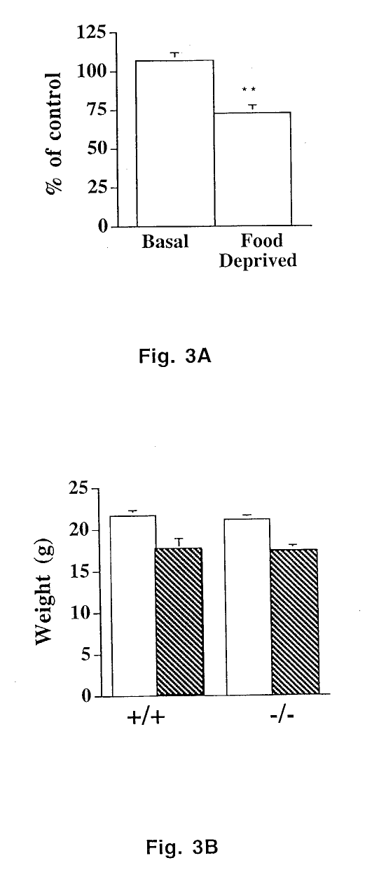

In order to determine if a stressful stimulus would alter the mutant animals' food intake, control and mutant mice were food deprived for 24 hrs and then refed, following which their food intake and weight changes were measured. Food deprivation results showed a significant decrease in food intake in the mutant mice following 24 hrs of food deprivation (FIG. 3A). Mutant mice consumed 75% of wild type food levels in the 24 hr period following the food deprivation. However, the mutant and wild type body weights were not significantly different following food deprivation or refeeding (FIG. 3B).

### EXAMPLE 7

Evaluation of Anxiety-Like Behavior in CRFR2 Deficient Mice in Elevated Plus Maze

Since CRFR1 mutant mice displayed anxiolytic-like behavior (8), CRFR2 null mutant mice were analyzed in similar tests. Control and mutant animals were evaluated using the elevated plus maze (EPM). Male mice between 22-24 weeks of age were used in this experiment. Littermate wild type mice were used as the controls. Animals were group housed, maintained under regular light/dark conditions (lights on 6:00 AM, lights off 6:00 PM), and handled on alternate days one week prior to testing.

The plus maze apparatus was made of black Plexiglas and had two open arms (30×5 cm) and two enclosed arms of the same size with walls 30 cm high. It was elevated 30 cm above the ground. The arms were connected by a central square (5×5 cm) and thus the maze formed a plus sign. A 25 watt lamp placed above the apparatus provided a 6 lux light level in the open arms. All testing was performed during the light phase of the light-dark cycle. Mice were habituated to the experimental room conditions for 1 hour prior to the behavioral testing and the subjects were individually tested in 5-min sessions.

Each mouse was placed on the center platform facing an open arm to initiate the test session. Behaviors scored were the number of open and closed arm entries and the amount of time spent on the various sections of the maze. Arm entries were defined as an entry of all four paws into the arm. Closed arm entries were taken as an index of locomotor activity in the plus maze. A camera mounted above the apparatus allowed the observation of animal behavior on a video monitor placed in an adjacent room. At the end of the test, the number of entries into and the time spent on the open arms were expressed as a percentage of the total number of arm entries and test duration, respectively. Results are expressed as the mean ± standard error of the mean. Behavioral parameters obtained from the EPM test were analyzed using the Student's t test.

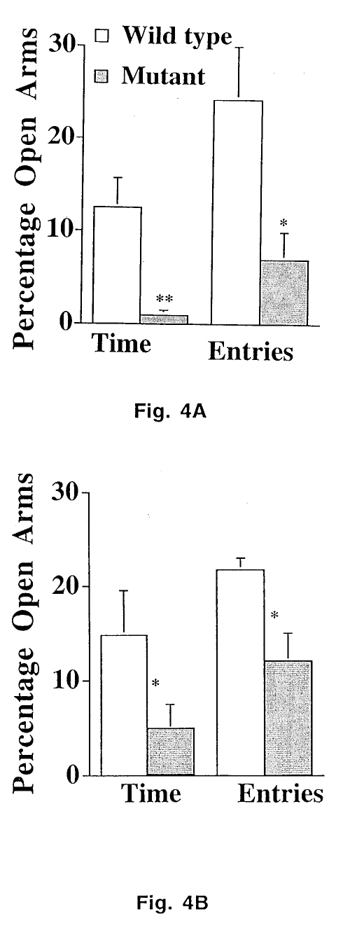

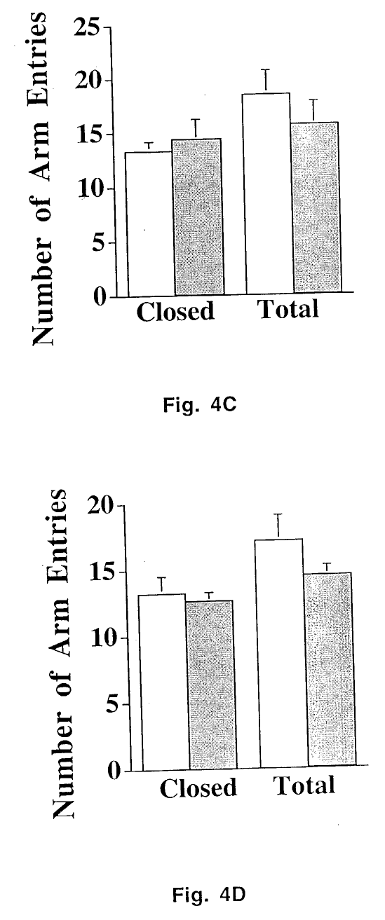

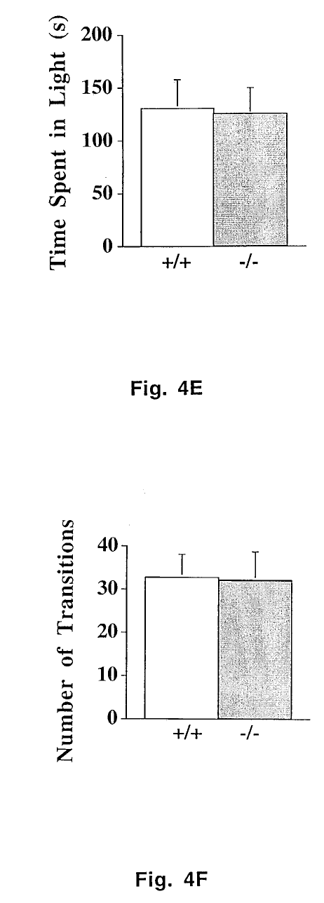

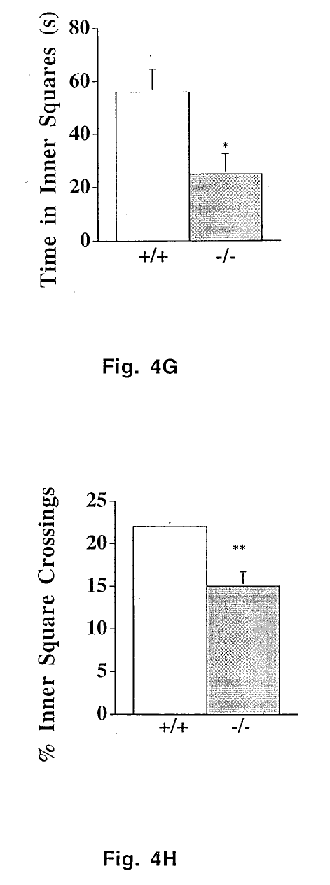

Results showed that CRFR2 null mutant mice spent less time on and entered less frequently the open arms of the plus-maze apparatus than did the wild type controls. A significant effect was found for both percent entries into the open arms [t (12)=2.684; p<0.02] and percent time in the open arms [t (12)=3.524; p<0.005] (FIG. 4A). The increase in anxiety-like behavior was not due to altered locomotor activity, as overall activity in closed arm [t (12)=0.469; p=0.64] and total arm entries [t (12)=0.904; p=0.38] was not different between the two groups (FIG. 4B). These results demonstrate that CRFR2 null mutant mice exhibit markedly increased anxiety-like behavior.

### EXAMPLE 8

Evaluation of Anxiety-Like Behavior in CRFR2 Deficient Mice in a Light/Dark Box

The behavior of CRFR2 null mutant and control mice was also analyzed for anxiety-like behavior in a light/dark box. A rectangular, plexiglass box was divided into two compartments, one painted white (28.5 cm×27 cm) and one painted black (14.5 cm×27.0 cm). Light intensity was 8 lux in the black compartment which was covered by a red plexiglass lid and 400 lux in the white compartment. The compartments were connected by an opening (7.5 cm×7.5 cm) located at floor level in the center of the partition. All testing was done during the dark phase of the cycle, between 19:00 hrs and 21:00 hrs. Each animal was tested for 10 min by being placed in the center of the white area and the number of transitions between the two compartments and the amount of time spent in the white area was recorded. A camera mounted above the apparatus allowed for observation and recording from an adjacent room.

Results from the Light/Dark box demonstrated that CRFR2 null mutant mice spent as much time in the light portion of the box and had as many transitions between the light and dark portions of the box as control mice (FIGS. 4C&D). No significant differences were detected between the two groups in this experiment.

### EXAMPLE 9

Effect of CRFR2 Deficiency on the Expression of Other Genes

As no gross anatomical defects were detected in components of the HPA axis (FIGS. 1D & 1E), the alterations in stress and behavioral responses in the mutant animals may be due to altered gene expression of other components of the CRF signaling pathway. To investigate this possibility, expression of UCN, CRF, and CRFR1 mRNAs were examined by in situ hybridization.

In situ hybridization was performed according to methods described previously (15). Briefly, tissue sections (20 μm) were fixed in 4% paraformaldehyde, rinsed in PBS, immersed in acetic anhydride, dehydrated through a series of graded ethanol, de-lipidated in chloroform, and again dehydrated. Slides were then hybridized with an ³⁵S-labeled riboprobe in a 50% deionized formamide hybridization mix overnight at 55° C. in a humidified incubation chamber. Following the incubation, slides were washed in 1×SSC at room temperature for 30 minutes with shaking, treated with 20 μg/ml RNase (Promega) at 37 C. for 30 min., rinsed in 1×SSC buffer at room temperature for 30 minutes, washed 3× for 20 minutes at 65 C. in 0.1×SSC with shaking, rinsed in 0.1×SSC at room temperature for 30 minutes, dehydrated in a series of graded ethanols, air dried, and apposed to Kodak hyperfilm (Eastman Kodak, Rochester, N.Y.) for three days.

After films were developed, slides were dipped in NTB2 liquid nuclear emulsion (Eastman Kodak; diluted 1:1 with water), exposed for 10 days, photographically processed, counter-stained with hematoxylin, and coverslipped. Slides were analyzed using the image analysis system Image Pro Plus (Media Cybernetics, Silver Springs, Md.). For analysis of the PVN and cAmyg, a circle tool (area=3022 pixels) was used to determine mean optical density for each section such that anatomically atlas matched sections for each animal were compared in the identical region of the PVN and cAmyg. The EW cell bodies expressing urocortin were too diffuse to analyze using standard optical density methods. Therefore, parameters were used such that the computer determined the number of cells within the designated EW expressing a minimum optical density by color and cell size as predetermined to exclude non-positive cells and background silver grains. Each cell determined to be positive by the computer for urocortin mRNA was then also counted for optical density. The average optical density and cell number for each section was then compared.

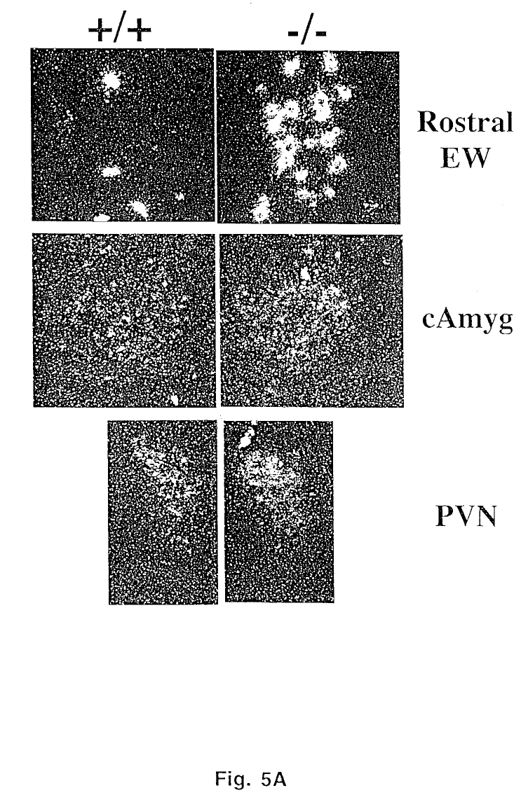

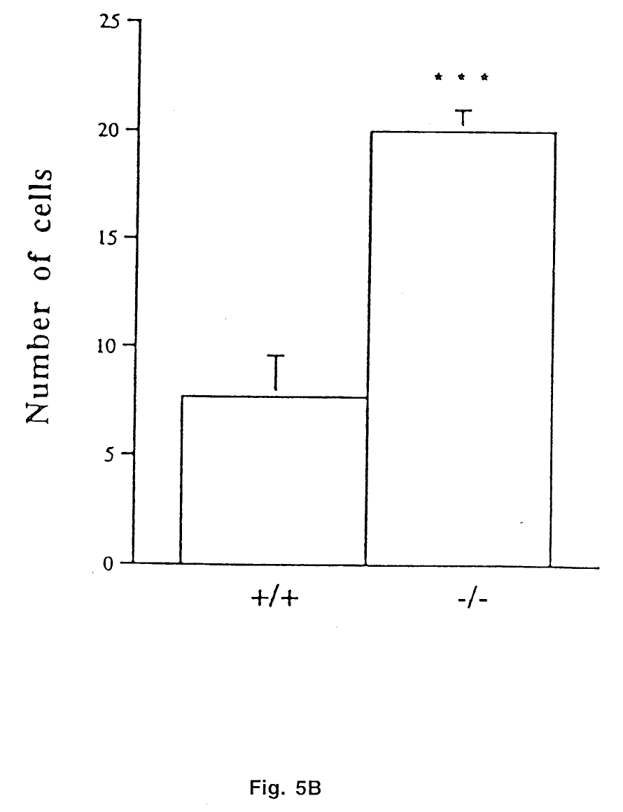

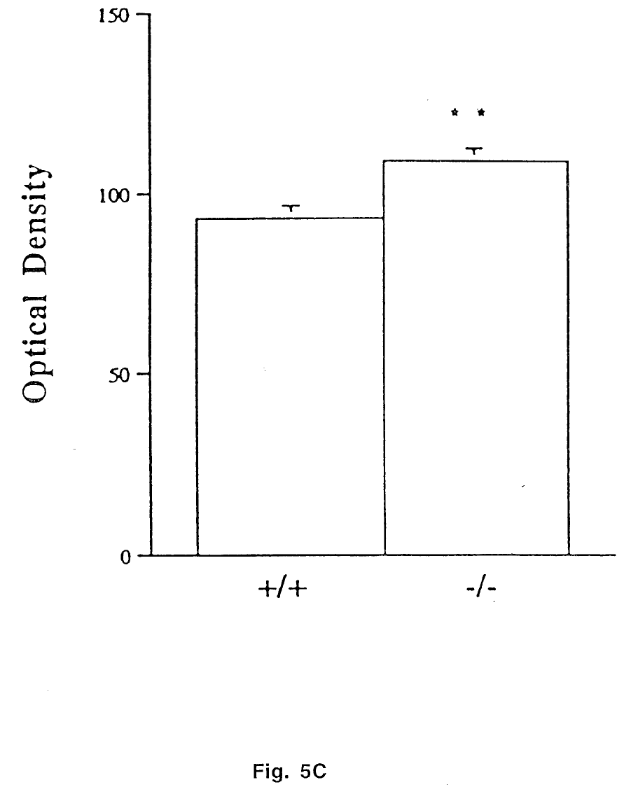

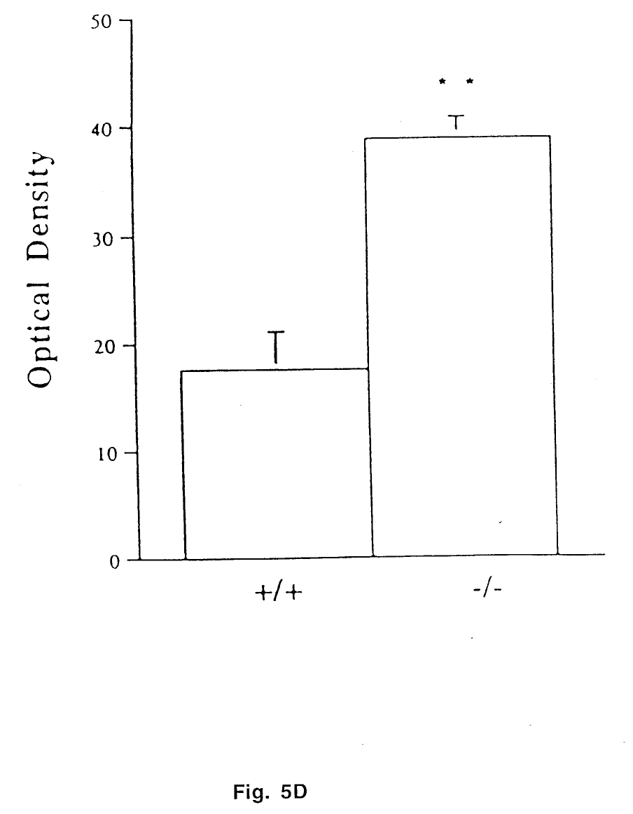

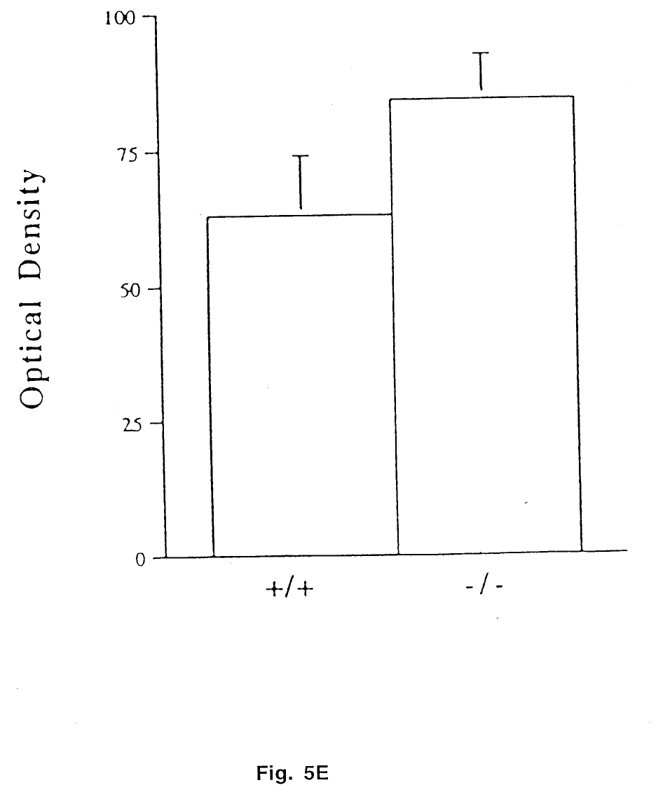

As illustrated in FIG. 5A, urocortin mRNA was significantly increased in the rostral region of the Edinger Westphal (EW) nucleus for both the number of cells expressing (FIG. 5B) as well as in the density of urocortin mRNA per cell (FIG. 5C) in the mutant animals. The central nucleus of the amygdala (cAmyg) showed a significant increase in CRF mRNA in the null mutant animals (FIGS. 5A & 5D). No significant change in CRF mRNA in the PVN was detected in basal, nonstressed animals (FIGS. 5A & 5E). The expression patterns or levels of CRFR1 mRNA in the brain or anterior lobe of the pituitary gland did not differ between the mutant and wild type mice (data not shown). These results show that CRFR2 null mutant mice have increased expression levels of CRF mRNA in the cAmyg and urocortin mRNA in the rostral Edinger Westphal nucleus.

### EXAMPLE 10

Hypotension in Response to UCN in CRFR2 Null Mutant Mice

Previous reports have shown hypotension in response to a peripheral injection of urocortin (2). Additionally, CRFR2s have been localized to the vascular endothelial cells (3, 7) and have been hypothesized to be responsible for the vasodilatory action of urocortin. In order to examine this, CRFR2 null mutant and control mice were injected with urocortin and the alteration in their blood pressure was measured.

The cardiovascular responses to intravenous infusion of urocortin and sodium nitroprusside, a vasodilator, were examined in mice (wild type: n=5; mutant: n=3) anesthetized with isofluorine. The arterial catheter for blood pressure recording was fabricated from a sterile PE-10 tubing softened and pulled to an outer diameter of 0.4 mm. The femoral artery was exposed, and the arterial catheter filled with heparin saline (500 U/ml) was implanted and secured with surgical threads and tissue glue (Vetbond). The catheter was connected to a blood pressure transducer (Statham), and the arterial pressure pulses were displayed on a Gould pen-recorder. A second catheter was then implanted in the external jugular vein for intravenous infusion of drugs. Drug infusion was performed 30 min following completion of the cannulation procedure. The venous catheter was connected to a drug-filled syringe. Infusion was completed within 0.5-1.0 min. Both wild type and mutants received an identical dose of urocortin (0.1 μg in 200 μl of 0.9% saline) and saline (as a control).

The doses used were determined from preliminary experiments with reference to data obtained from corresponding studies in Sprague Dawley rats (2). In order to verify that the lack of cardiovascular response to the urocortin injection in mutants was not attributed to the loss of ability of the mice to vasodilate, the mutant mice also received a second infusion of sodium nitroprusside (0.8 μg in 100 μl of 0.9% saline) following recovery of arterial pressure from the urocortin infusion. The mean arterial pressure (MAP) was determined from the blood pressure tracings.

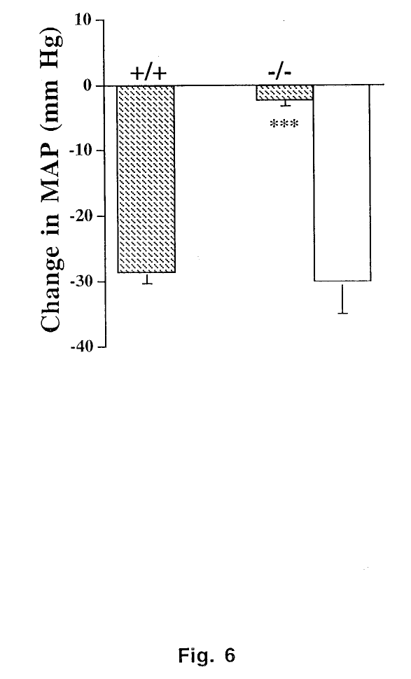

Intravenous infusion of urocortin (0.1 μg) resulted in a prominent depressor response (−28.3±2.0 mm Hg) in control mice (FIG. 6). The reduction in arterial pressure persisted throughout the recording period (90-120 min). In stark contrast, the mutants showed no measurable responses to urocortin (only 1 mutant mouse examined showed a very small and transient reduction (−3.5 mmHg) in arterial pressure which is likely attributable to the injection pressure itself) (FIG. 6). In order to verify that the peripheral vasculature of the mutants was able to vasodilate in response to another stimulus, sodium nitroprusside (NP), which causes vasodilation as a nitric oxide donor, was administered to mutant mice. A rapid and robust depressor response was consistently observed in response to the sodium nitroprusside injection (−30.0±5.0 mm Hg).

### EXAMPLE 11

Summary of Effects of CRFR2 Deletion on anxiety and Stress

The results presented here suggest that the CRFR2 null mutant mice display a stress-sensitive and anxiety-like phenotype. Although basal feeding and weight gain were normal, mutant mice responded to food deprivation by consuming less food following the stress of food deprivation. While this may be an effect of metabolism, it is possible that the stress of food deprivation alters the anxiety state of the animal thus decreasing their appetite. The mutant mice also displayed a rapid HPA response to restraint stress, again suggesting that these animals are more sensitive to stress. The decrease in ACTH levels in the mutants observed following ten minutes of restraint may be the result of a more rapid negative glucocorticoid feedback on the hypothalamus, since the mutant mice showed higher steroid levels earlier than the control mice. Taken together, the feeding and HPA axis results suggest a hypersensitivity to stress in the CRFR2 null mutant mice, although one can not rule out other physiological explanations for either the altered feeding response or the increased rate in which the HPA axis in the mutant mice responds to stress.

The mutant mice also display increased anxiety-like behavior in the EPM. However, these mice show similar levels of anxiety-like behavior in the light/dark box. Although pharmacological sensitivity and specificity has generally been demonstrated across many animal tests of anxiety, task differences are sometimes observed (16, 17). While both are classified as unconditioned exploration tests, the light/dark box measures neophobia in addition to exploration. Performance in the EPM is determined by exploration of aversive environments (18). Light conditions during testing can also significantly influence the ability to detect anxiolytic or anxiogenic effects in animal tests (16). This profile of results for the CRFR2 null mutant mice demonstrates heightened emotionality related to exploration of aversive environments but not neophobia. Previous reports have shown that mice deficient for neuropeptide Y (NPY) display a similar behavioral phenotype, normal in the light/dark box but anxious in the EPM (19). These NPY mutant mice were classified as being anxious which supported previous findings that an injection of NPY decreased anxiety (20). The results obtained with the CRFR2 null mutant mice demonstrate that the EPM may be a more sensitive task for detecting the anxiety in these mice.

### EXAMPLE 12

Possible Effects of Increased CRF in cAmyg on Anxiety

Increased CRF mRNA in the cAmyg may explain the anxiety-like behavior and increased HPA axis sensitivity of the mutant mice, since this nucleus expresses CRFR1 (7) and plays a major role in transduction of stress signals (21). In addition, the septum which contains an abundance of CRFR2 has been shown to modulate the activity of the amygdala (22-24) and lesions of this nucleus result in decreased ACTH secretion following restraint stress (25-28). Therefore, it is possible that during stress CRFR2 in the lateral septum modulates activity of the amygdala, and in the absence of CRFR2, unimpeded amygdala activity may result in a rapid HPA response and increased anxiety-like behavior.

Lesions of the amygdala have been shown to block CRF-induced anxiety (21) as well as hyperemotionality resulting from septal lesions (22). This neural pathway may explain the decreased anxiety-like behavior seen in the CRFR1 deficient mice (8) as well as the increased anxiety-like behavior in the CRFR2 deficient mice. Therefore, the CRFR2 null mutant mouse provides possible evidence for a novel mechanism of receptor modulation in anxiety-like behavior.

### EXAMPLE 13

Possible Mechanisms for Anxiety Caused by Increased UCN mRNA in the Rostral EW

Increased urocortin mRNA in the rostral EW may be a second mechanism leading to increased anxiety-like behavior in the mutant mice, since urocortin has been shown to induce anxiety-like behaviors when injected intravenously (29). The rostral EW projects to many regions in the CNS including the locus coeruleus (LC) (30) and injection of the urocortin-related molecule, CRF, into the locus coeruleus results in an anxiety-like response (31). Thus, increased urocortin mRNA in the rostral EW may activate the locus coeruleus to elevate anxiety-like responses and/or hypersensitivity to stress.

### EXAMPLE 14

CRFR2 Null Mice and the Sensitivity of the Autonomic Nervous System

Additional explanations for the increased anxiety-like behavior, such as heightened sensitivity of the autonomic nervous system (32-34), cannot yet be ruled out. Previous studies using antisense oligonucleotides have found conflicting results regarding the role of CRFR2 in anxiety and behavior (35, 36).

Although these reports show an anxiolytic-like effect by injection of CRFR1 antisense oligonucleotides, neither study reported consistent findings regarding injection of the CRFR2 antisense oligonucleotides. While the technique of antisense oligonucleotide injection offers potential promise, it remains under scrutiny since decreased levels of protein cannot be substituted for complete elimination of the target, as is accomplished in a knockout animal.

### EXAMPLE 15

Effect of UCN on Vasodilation Confirmed

Absence of CRFR2 in the null mutant mice allowed for confirmation of the effect of urocortin on vasodilation. Mutant mice had no response to intravenous urocortin, while wild type animals showed a dramatic decrease in mean arteriole pressure. Injection of nitroprusside resulted in vasodilation in the mutants, thus confirming that the lack of response to urocortin was not due to a physical inability of the mutant vasculature to dilate, but specifically to the absence of CRFR2. These results support the hypothesis that the effect of urocortin on hypotension (2, 14) occurs via action at CRFR2 in the vascular endothelial cells (3, 7), since the CRFR2 null mutant mice showed no response to urocortin. Although the physiological stimulus under which UCN-induced vasodilation would most likely occur is not currently known, the effect of urocortin on CRFR2 in the vasculature may be an interesting target in drug development for hypertension.

In summary, these results demonstrate that CRFR2 deficient mice exhibit increased anxiety-like behavior in an elevated plus maze and a hypersensitive HPA axis in response to stress. CRFR1 and CRFR2 null mutant mice provide valuable models of anxiety and depression and may further help delineate the molecular mechanisms underlying these diseases. Study of the CRF signaling pathway and its role in the management of anxiety and depression may provide the necessary clues required for the effective treatment of these diseases.

### EXAMPLE 16

Angiogenesis is Stimulated in CRFR2 Null Mutant Mice

The CRFR2 null mutant mice appeared to exhibit an increase in the size and number of blood vessels in various tissues. Since the CRFR2 receptor and its activity have been localized within the endothelial cell layer of blood vessels (3, 7), it was hypothesized that CRFR2 may play a role in regulating angiogenesis. To confirm that CRFR2 null mutant mice had an increased number of blood vessels of larger size, tissues from control and CRFR2 null mutant mice were immunostained with an antibody against platelet-endothelial cell-adhesion molecule (PECAM), a blood vessel specific marker.

Tissues were obtained from the anterior pituitary, white adipose tissue, and dorsal brain surface of both control and CRFR2 null mutant mice of 3-4 months of age. After the tissues were fixed in 4% paraformaldehyde for two days, the tissues were bleached in Dent's fix (4:1 methanol:DMSO) plus 5% hydrogen peroxide overnight. The tissues were washed 3 times in 1× TBS with 1% Tween-20 for 30 minutes each and blocked overnight with 5% goat serum in dilution buffer (0.5 M NaCl, 0.01 M PBS, 3.0% BSA, and 0.3% Triton X-100) plus 1% DMSO at room temperature. On the following day, a 1:1000 dilution of anti-PECAM antibody (Pel Freeze) was added to the blocking mix, which was then incubated for another 2 days at room temperature. The antibody was removed and the tissues were washed 3 times for 1 hour each with 1× TBS plus 1% Tween-20 and 1% DMSO. Goat anti-RAT HRP secondary antibody was added at a 1:5000 dilution and allowed to incubate overnight at room temperature. The tissues were washed as above and a final wash in 1× TBS alone was performed for 1 hour at room temperature. The peroxidase reaction was carried out in the presence of glucose oxidase-containing (Calbiochem) reaction mix until an orange-brown color developed. The tissues were dehydrated in a graded methanol series and cleared with glycerol.

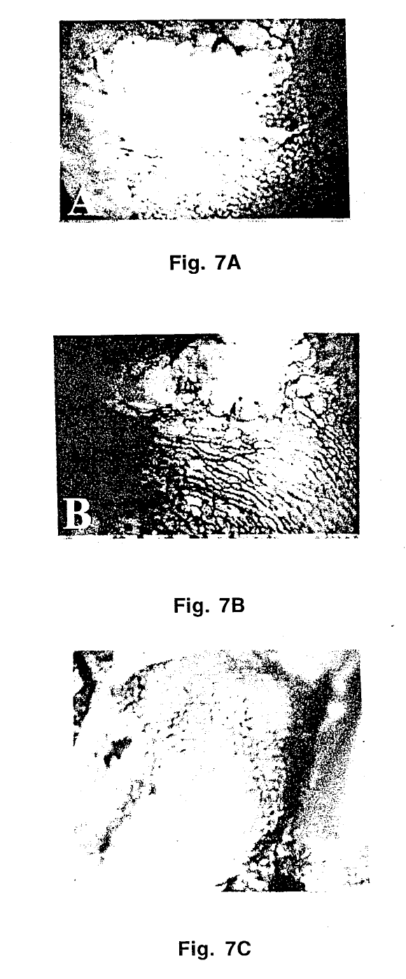

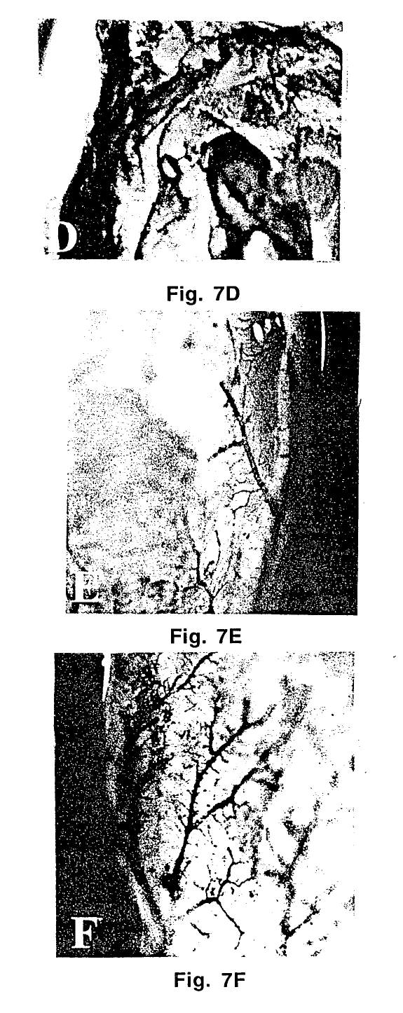

The results of the anti-PECAM immunostaining are shown in FIGS. 7A-7F. These experiments confirmed that the absence of the CRFR2 receptor in the null mutant mice results in an increase in number and size of blood vessels in the anterior pituitary (FIG. 7B), white adipose tissue (FIG. 7D) and dorsal brain surface (FIG. 7F). The same tissues in control mice are shown in FIG. 7A-anterior pituitary; FIG. 7C—white adipose tissue; and, FIG. 7E—dorsal brain surface. Therefore, one of the roles of the CRFR2 receptor in normal mice is to mediate a CRF-induced inhibition of angiogenesis.

### EXAMPLE 17

CRFR2 Has No Effect on Angiogenesis in Embryonic Mice

To determine whether the CRFR2 receptor may be involved in blood vessel formation during embryonic development, anti-PECAM immunostaining experiments were performed on tissue sections from day 11 embryonic mice. Sections of tissues from embryonic mice were prepared and treated in the same manner as the sections from adult mice.

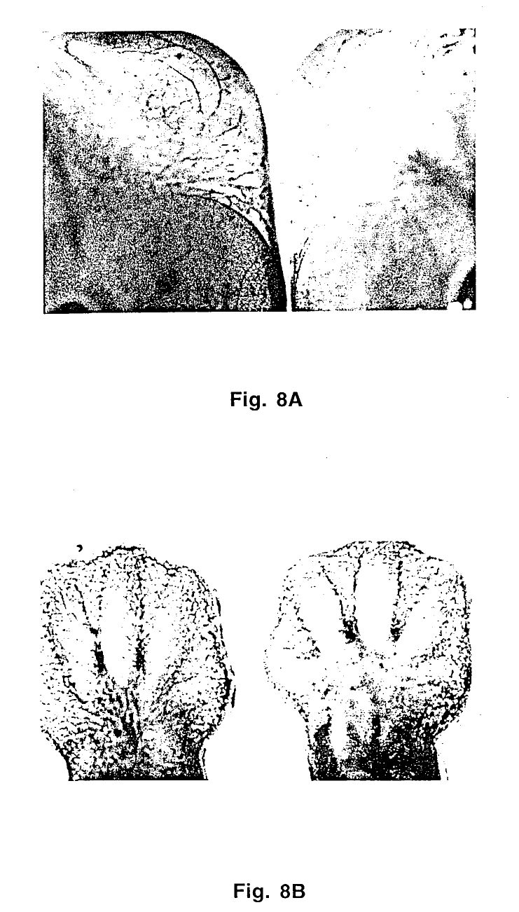

FIG. 8A shows anti-PECAM immunostained sections from the heads of CRFR2 null mutant (right) and control (left) mice, while FIG. 8B shows immunostained sections from the front paws of CRFR2 null mutant (right) and control (left) mice. No difference in vessel number or size was observed between CRFR2 null mutant mice and control mice in either the head or front paw tissue sections. Thus, CRFR2 appears to be involved in angiogenesis only in fully developed mice.

### EXAMPLE 18

Microfil Polymer Characterization of Vascularization in Adult Mice

To further characterize the hypervascularization of CRFR2 null mutant mice, the vascular tissues of control and mutant mice were perfused with microfil polymer to confirm that an increase in vessel volume had occurred. In preparation for perfusion, a 30 gauge needle was placed in the left ventricle of anesthetized adult or three week old CRFR2 null mutant and control mice. The perfusion was performed with a syringe pump until the perfusate drained freely from a drain vent opened in the right atrium for that purpose. The animals were placed at 4° C. overnight to allow the polymer to cure. Tissue sections were dissected from the cured animals and dehydrated through a graded ethanol series starting with 25% ethanol on day one. After bleaching with 6% hydrogen peroxide on day 2, the ethanol series was continued with 50% ethanol followed by 75% ethanol on day 3, 95% ethanol on day 4, and 100% ethanol on day 5. The tissues were then cleared in glycerol prior to analysis. Following removal of the soft tissue, the volumes of the vascular beds of various tissues could be observed.

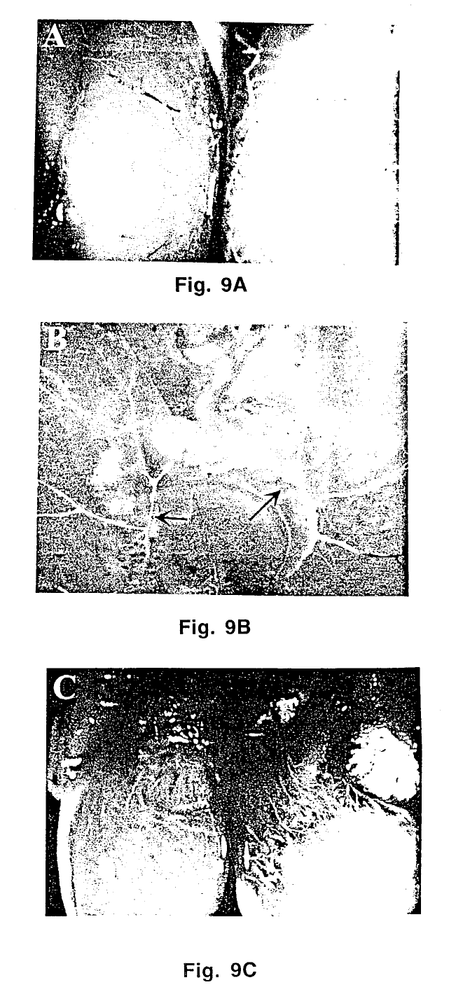

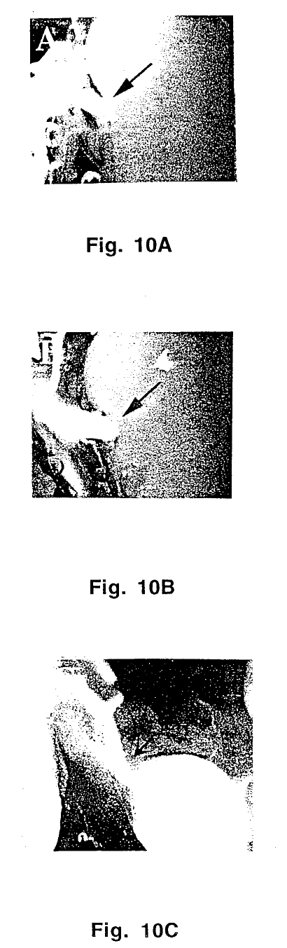

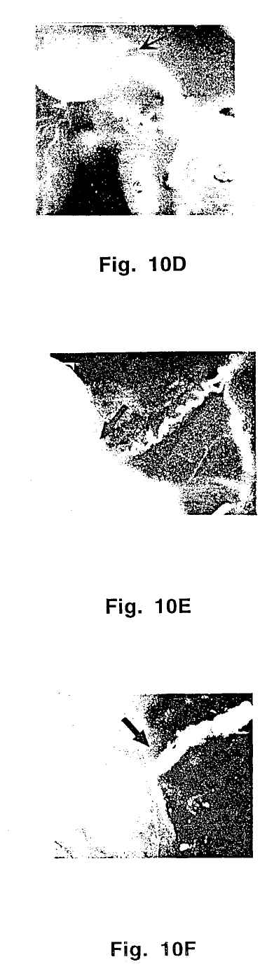

FIGS. 9 and 10 show microfil perfused tissues from adult CRFR2 null mutant and control mice. In FIG. 9, the tissue from the normal mouse is shown on the left side of each panel while a similar section from a CRFR2 null mutant mouse is shown on the right side. Increased vessel size and number are observed in all tissues from CRFR2 null mutant mice including the dorsal brain surface (FIG. 9A), large intestine (FIG. 9B) and heart (FIG. 9C). In FIG. 10, the primary arteries for the kidney (FIGS. 10A and 10B), adrenal glands (FIG. 10C and FIG. 10D) and testis (FIGS. 10E and 10F) are indicated with arrows. The major vessels of the CRFR2 null mutant (FIG. 10B, FIG. 10D and FIG. 10F) mice are significantly increased in size relative to those of the control mice (FIG. 10A, FIG. 10C and FIG. 10E). These results, combined with the anti-PECAM immunostaining results, confirm that mice deficient for CRFR2 exhibit increased hypervascularization in all tissues observed including the brain, heart, pituitary gland, and gastrointestinal tract. Both the size and number of blood vessel was increased.

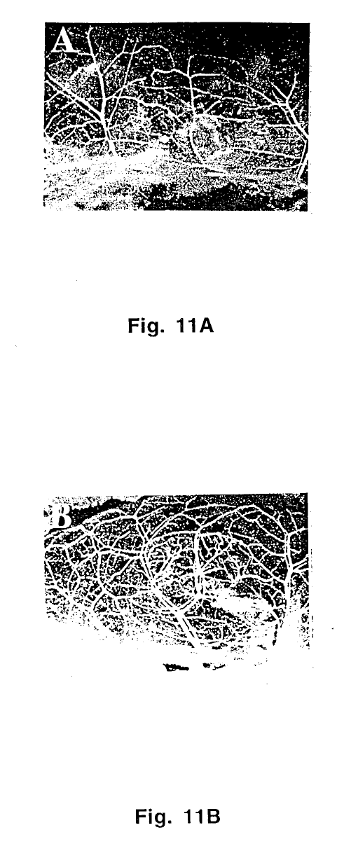

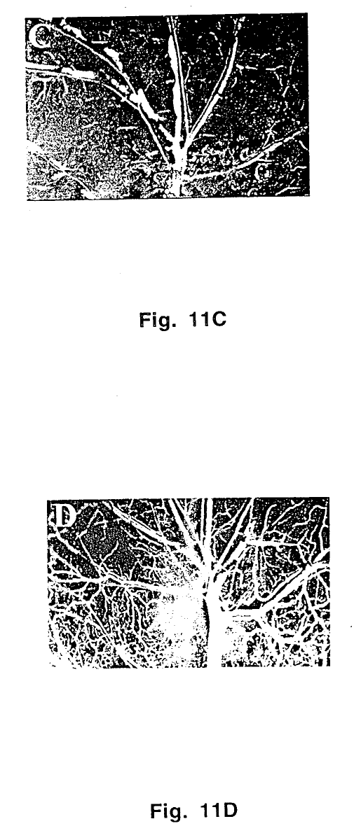

Microfil perfused tissues from 3 week old mice are shown in FIGS. 11A-11D. The CRFR2 null mutant mice exhibit an increase in the number of blood vessels in the small intestine (FIG. 11B vs. FIG. 11A) and stomach (FIG. 11D vs. FIG. 11C). These results suggest that hypervascularization first increases the number of blood vessels and the blood vessels increase in size as the mouse ages.

### EXAMPLE 19

Analysis of VEGF Expression in CRFR2 Null Mutant Mice

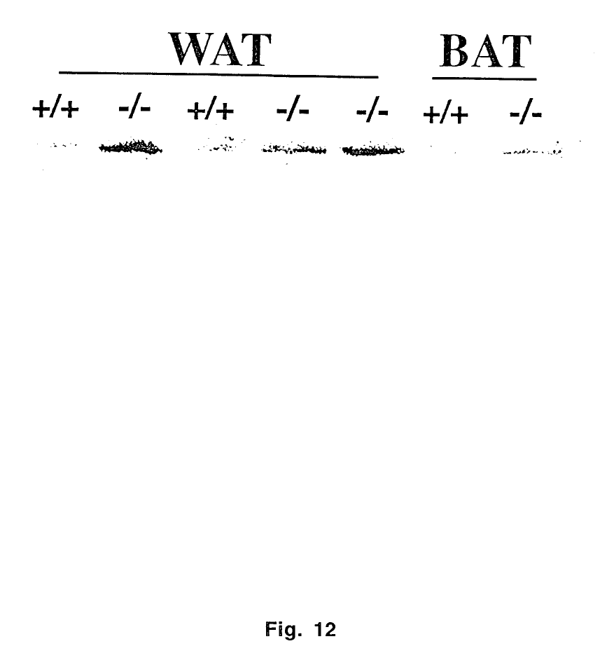

To determine if CRFR2 has an effect on vascular endothelial growth factor (VEGF) expression, tissues from CRFR2 null mutant and control mice were examined by western blot analysis for VEGF content. White (WAT) and brown (BAT) adipose tissues were homogenized in buffer (50 mM TrisHCl, pH 7.4, 1 mM DRR, 2 mM MgCl2, 1 mM EDTA, 0.5 mM PMSF, 5 μg/ml leupeptin, 2 μg aprotinin). 40 μg aliquots of the protein extracts were separated on a 10% SDS-PAGE gel (Novex, San Diego) and transferred to nitrocellulose membranes. The blots were blocked in 5% nonfat dry milk for 1 hour and washed with 1× TBS plus 0.2% Tween-20 (TBST). The blots were then incubated with a 1:1000 dilution of anti-VEGF antibody for 1 hour, washed twice in TBST for 20 minutes each, and incubated with a 1:10,000 dilution of anti-rabbit HRP for 1 hour. After being washed twice in TBST for 20 minutes each time, the blots were visualized with ECL reagent. A representative blot is shown in FIG. 12. Increased VEGF expression was observed in all tissues examined from CRFR2 null mutant mice, indicating a possible interaction between CRFR2 and VEGF production.

### EXAMPLE 20

Hair Growth is Stimulated in Urocortin Treated Mice

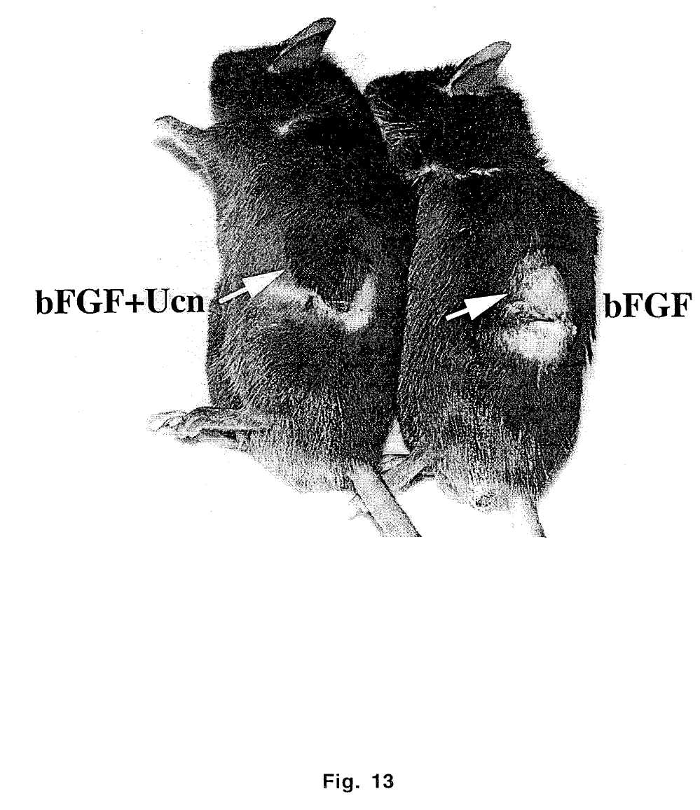

Small regions area of mice were shaved over of their skin and gel foam sponges impregnated with bFGF and urocortin, various growth factors, and CRF antagonist astressin were surgically implanted under the shaved skin. In the mice implanted with both bFGF and urocortin, substantial hair growth in the area immediately above the implanted sponge was observed after only five days. Little hair growth was observed in the shaved area of mice implanted with sponges containing only growth hormones or astressin. FIG. 13 shows the hair growth in a mouse implanted with urocortin and bFGF as compared to a mouse in which only bFGF was implanted. Therefore, urocortin and bFGF stimulate rapid hair growth.

### EXAMPLE 21

Abnormal Homeostatic Responses of CRFR2-Deficient Mice to Challenges of Increased Dietary Fat and Cold

CRFR2-mutant and wild-type mice were housed under a 12-hour light/dark cycle. All studies were done according to experimental protocols approved by the Salk Institute Institutional Animal Care and Use Committee, and all procedures were conducted in accordance with institutional guidelines.

All data are presented as means ±SEM and were evaluated by two-way ANOVA for repeated measures, followed by Fisher's protected least significant difference post hoc test, using StatView SE+ (Abacus Concepts, Berkeley, Calif.). P<0.05 was defined as statistically significant.

### EXAMPLE 22

Lower Body Fat but Higher Food Intake on High-Fat Diet

Individually housed CRFR2-mutant and wild-type male mice were fed a high-fat (58%) or low-fat (11%) diet (Research Diets, Inc.) ad libitum (n=7) for 16 wk. By calories, the low-fat diet contained 60% corn starch and 7% hydrogenated coconut oil, whereas the high-fat diet contained 13% corn starch and 54% coconut oil. Both diets contained 12% maltodextrin, 4% soybean oil, 16% casein, and identical vitamins and minerals. Food intake and body weight was measured 3 times per week during the 16-wk study. Preweighed food pellets were placed in the hopper, and, to allow for accurate food measurements, minimal bedding was used in the cage to allow for retrieval of all food pieces for weighing. Plasma samples were taken at the end of the study for lipid measurement. Carcasses were immediately frozen on dry ice. Carcasses and plasma samples were shipped to the University of Alabama at Birmingham for analysis. Feed efficiency is calculated as: gram weight gained per gram food consumed.

For measurements of body composition and plasma lipids, carcasses were thawed at room temperature and the gastrointestinal tract removed (stomach, small and large intestine, and cecum) leaving the eviscerated carcass. Body water content was determined by drying the eviscerated carcass to a constant weight in a 60° C. oven. The dried eviscerated carcass was then cut into small pieces, ground to a homogeneous mixture, and extracted with petroleum ether in a Soxhlet apparatus to determine fat mass and fat-free dry mass. Fat-free dry mass was then combusted overnight at 600° C. (8 hours minimum) to determine eviscerated carcass ash. Plasma triglycerides and cholesterol were measured with an Ektachem DTII System (Johnson & Johnson Clinical Diagnostics, Rochester, N.Y.) in 10 μl plasma. Free fatty acids were assayed with nonesterified fatty acid-C reagents obtained from Wako Diagnostics (Richmond, Va.) in which the assay was modified for use with 10 μl plasma.

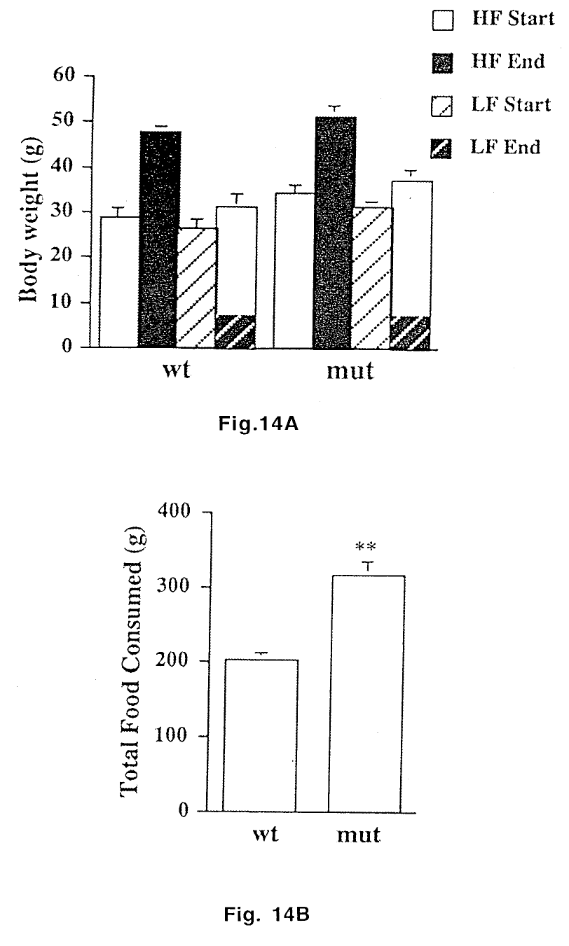

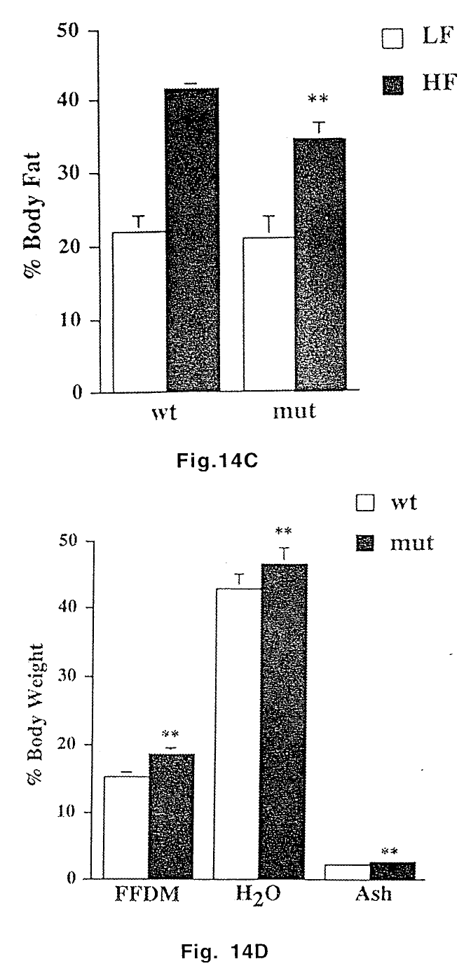

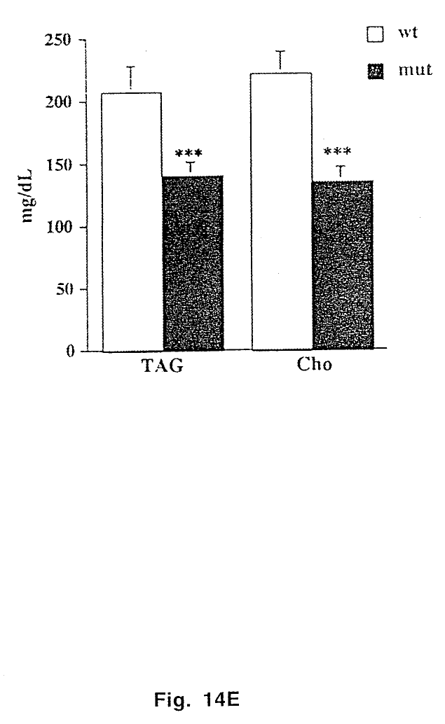

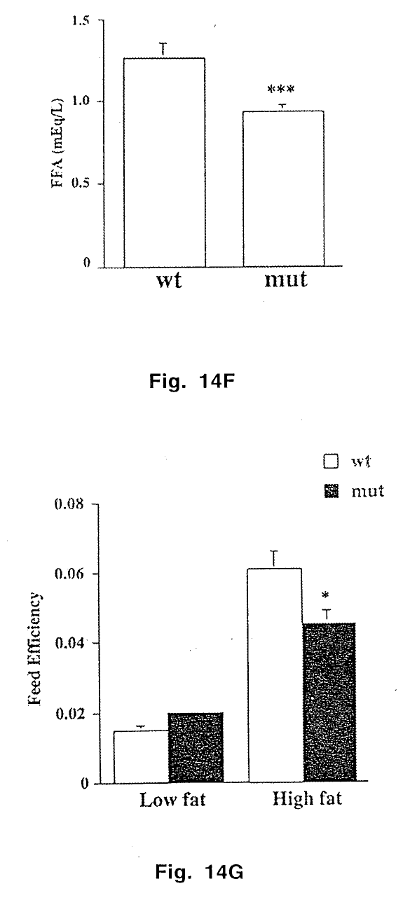

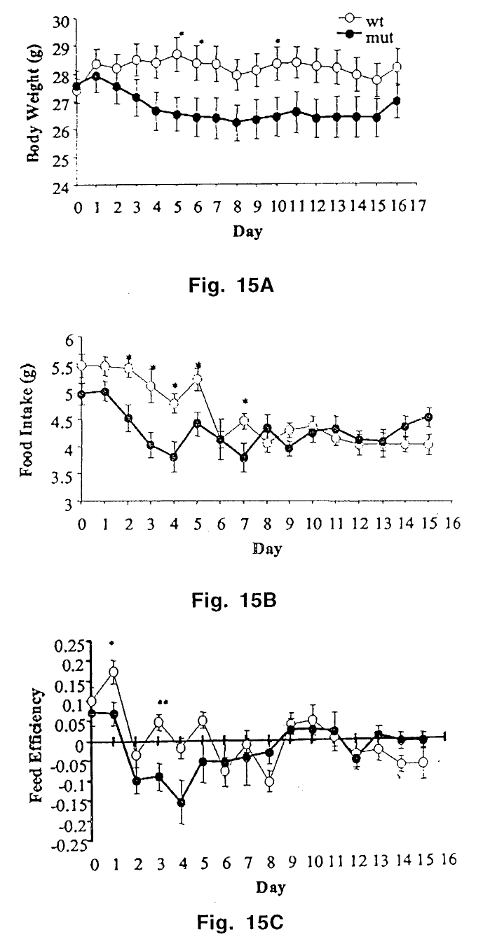

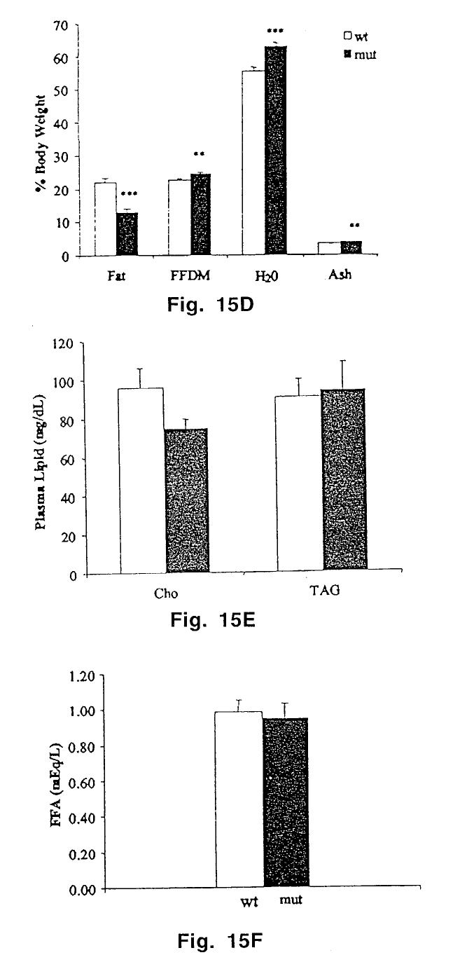

To determine the homeostatic responses of a high-fat challenge, mice on either a high-fat or a low-fat diet were monitored for food intake, weight gain, and body composition. Although both genotypes gained similar weight on the high-fat diet, the mutant mice consumed significantly more food than did the wild-type mice (FIGS. 14(A-B)). No difference was found between genotypes for food consumption while on the low-fat diet (wt=257.2±27, mut=287.0±2). Body composition analysis indicates that the mutant mice, despite consuming significantly more high-fat food during the study, had significantly less body fat than wild-type mice (FIG. 14C). Because the overall end body weight following the high-fat diet study was similar for mutant and wild-type mice, the difference in composition for the mutant mice was compensated by slightly nmore water, bone (ash), and muscle (fat-free dry mass (FFDM-ash)) than wild-type mice (FIG. 14D). The same differences in terms of absolute values were found for mice on the high-fat diet (fat: wt=19.03 g, mut=15.38 g; FFDM: wt=7.0 g, mut=8.76 g.). Percentage of body fat was not different between genotypes while on the low-fat diet (FIG. 14C). Serum triglyceride, cholesterol, and free fatty acid levels were also significantly lower in the mutant mice despite the increased high-fat food intake (FIGS. 15(E-F)). No significant differences were detected for plasma lipids between genotypes while on the low-fat diet (triglycerides: wt=111.2±22, mut=101.8±10; cholesterol: wt=105.3±10, mut=76.4±9; free fatty acids: wt=0.63±0.08, mut=0.57±0.07). CRFR2-mutant mice had a lower feed efficiency, compared with wild-type mice following 16 wk of high-fat diet (FIG. 14G). No differences in feed efficiency were found between genotypes on the low-fat diet.

### EXAMPLE 23

Increased Sensitivity to Repeated Cold Stress

Wild-type and CRFR2-mutant mice (n=10) were individually housed for 2 weeks before testing. Mice were given a 4-day basal period to adjust to food pellets (standard chow) on the cage floor and to being handled. The experiment was conducted for a total of 15 days. During both basal and cold stress periods, weight gain and food consumption were measured daily at 1500 hours. Mice were exposed to cold (4° C.) for 1 hour daily at 1545 hours. The apparatus used for the cold stress was as follows: Two 50-gallon coolers were modified to each hold a rack of 10 containers. Each container was 13 cm deep with a diameter of 9 cm (each lid had five small air holes). Each container with lid housed one mouse, which was submerged and completely surrounded by an ice-water slurry, 6 cm from the bottom of the chest. The containers were numbered and housed the same mouse each time throughout the duration of the experiment. Extreme care was taken to prevent the mice from getting wet. The temperature per chest was recorded as the temperature of the air inside the container. In each of the two chests, five wild-type and five CRFR2-mutant mice were distributed alternately throughout the 10-container rack within the chest. Immediately following the 1-hour cold stress, mice were returned to their repective cages containing preweighed fresh food. The containers and racks were washed and air dried overnight. For measurement of body temperature following cold stress, a rectal probe thermometer was used (n=6) (Harvard Apparatus).

Mutant mice exposed to repeated cold stress lost significantly more weight during the 15-day study and consumed significantly less food during the first half of the study than wild-type mice did (FIGS. 15(A-B)). Feed efficiency was calculated on a daily food consumption basis and found to be lower for the mutant mice during the first portion of the study (FIG. 15C). Body composition analysis, similar to the high-fat diet study, showed that the mutant mice had significantly lower body fat than wild-type mice following the cold stress despite overall body weights being similar at the end of the study (FIG. 15D). As before, the difference in body composition for the mutant mice was compensated by slightly more water, bone, and muscle than wild-type mice (FIG. 15D). No significant differences were detected for cholesterol, triglycerides, or free fatty acid levels (FIGS. 15(E-F)).

No difference between genotypes was detected for body temperatures before or after cold stress (wild-type basal 36.3±0.38° C., CRFR2-mutant basal 36.0±0.21° C.; wild-type following 1 hour cold=37.8±0.18° C., CRFR2-mutant following 1 hour cold=37.6±0.07° C.).

### EXAMPLE 24

Glucose and Insulin Responses

In the glucose and insulin challenge tests, individually housed male CRFR2-mutant and wild-type mice (on standard chow) were fasted overnight (dark cycle) before glucose or insulin challenge. Glucose (2 g/kg in saline) was administered intraperitoneally, and tail blood was collected at 0 min (before ip injection), 5, 30, and 60 min after the injection. Glucose was measured immediately using the Lifescan One Touch glucometer. For insulin tolerance, mice were ip injected with insulin (0.75 U/kg, Sigma, St. Louis, Mo.) and blood glucose measured at 0 min (before ip injection) and 5 and 60 min following the injection. For the high-fat diet, mouse basal glucose and insulin levels were measured before the start of the diet and following 4 wk on the high-fat diet (as described in Example 22), following an overnight fast.

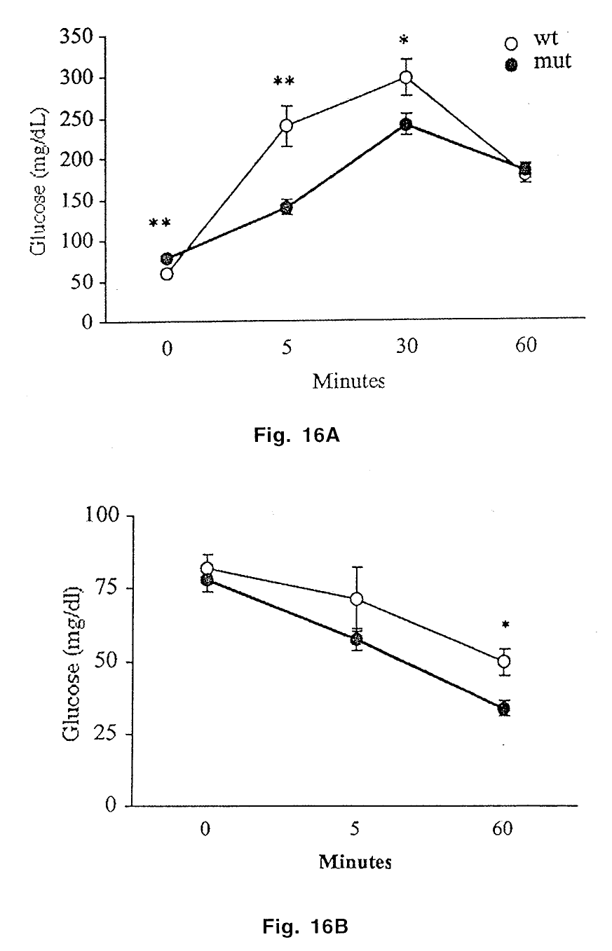

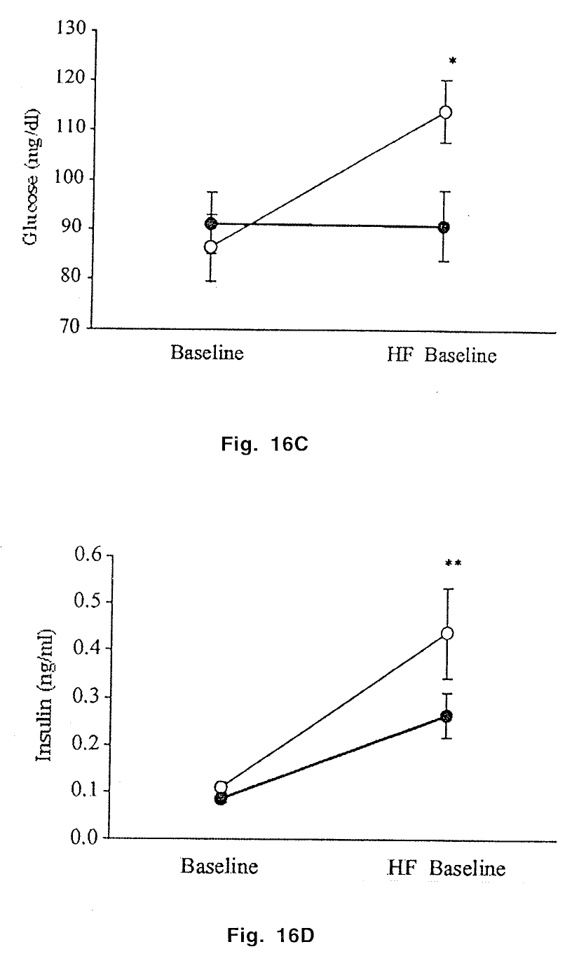

Plasma glucose levels in response to glucose and insulin challenges were compared in mutant and wild-type mice. CRFR2-mutant mice demonstrated lower peak plasma glucose levels following a glucose challenge (FIG. 16A). Glucose levels in mutant mice declined more rapidly following an insulin challenge (FIG. 16B). Following 4 wk on a high-fat diet, mutant mice showed no elevation in their plasma glucose levels, compared with their baseline and only a slight rise in their plasma insulin levels, compared with wild-type animals (FIGS. 16(C-D)).

### EXAMPLE 25

Abnormal Adipose Cell Size and Elevated UCP1

Tissue histology was performed on white adipose tissue (WAT) and BAT from male mice 16-20 weeks of age (on standard chow); the tissues were fixed in neutral-buffered formalin (Sigma) for 48 hours, dehydrated in 70% ethanol, and paraffin embedded. Tissues were sectioned at 8-μm thickness, deparaffinized, and stained with hematoxylin and eosin.

Western blot analyses were performed for comparison of UCP1 levels. BAT tissues were taken from control and CRFR2-mutant male mice under basal conditions during the morning hours. Tissues were homogenized in buffer (50 mM Tris pH 7.4, 1 mM dithiothreitol, 2 mM MgCl₂, 1 mM EDTA, 0.5 mM phenylmethylsulfonyl fluoride, 5 μg/ml leupeptin, 2 μg/ml aprotinin). Protein extracts (40 μg/lane as determined by Bradford assay for protein content) were separated by 10% SDS-PAGE (Novex, San Diego, Calif.) and transferred to a nitrocellulose membrane. Blots were blocked in 5% nonfat dry milk for 1 hour, washed in 1× Tris-buffered saline (TBS) plus 0.2% Tween-20 (TBST), incubated with anti-UCP1 antibody (1:1000) (Calbiochem, La Jolla, Calif.) for 1 hour, and washed in TBST 2×20 min. Blots were visualized with enhanced chemiluminescence reagent (Amersham).

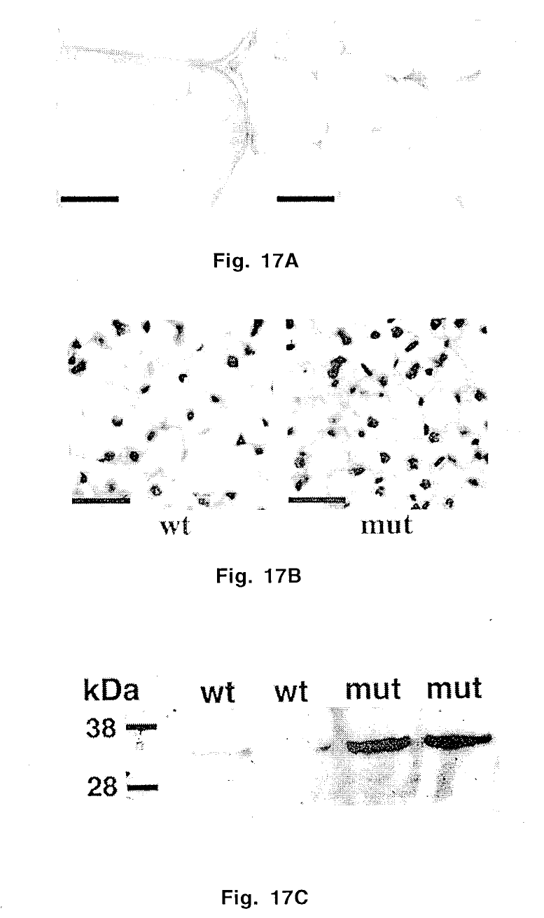

To examine indices of sympathetic tone, BAT and WAT tissues from CRFR2-mutant and wild-type littermate male mice were analyzed. Additionally, protein levels of BAT UCP1 were compared in these mice. CRFR2-mutant mice have smaller WAT (FIG. 17A) and BAT cell size (FIG. 17B). The WAT from the wild-type mice is composed of large, polygonal cells with prominent griglyceride depots, whereas adipocytes from mutant mice appear smaller and more rounded with diminished triglyceride stores. Western blot analysis showed that basal BAT UCP1 levels were substantially elevated in the CRFR2-deficient mice, compared with wild-type nice levels (FIG. 17C).

### EXAMPLE 26

No Significant Differences in 24-Hour Basal Locomotor Activity

Locomotor activity of male wild-type and CRFR2-mutant mice 6 months of age was examined across 24 hours (n=4). Testing took place in Plexiglas cages (42×22×20 cm) placed into frames (25.5×47 cm) mounted with two levels of photocell beams at 2 and 7 cm above the bottom of the cage (San Diego Instruments, San Diego, Calif.). These two sets of beams allowed for the recording of both horizontal (locomotion) and vertical (rearing) behavior. A thin layer of bedding material was applied to the bottom of the cage. Food pellets were scattered evenly across the bottom of the cage, and a waterspout was extended down into the cage just above the level of the vertical beams. Mice were placed in the activity boxes for the final 3 hours of their light (inactive) cycle to habituate them to the testing environment. Immediately following this habituation test, mice were tested for 24 hours, including a standard 12 hour dark and a 12 hour light phase.

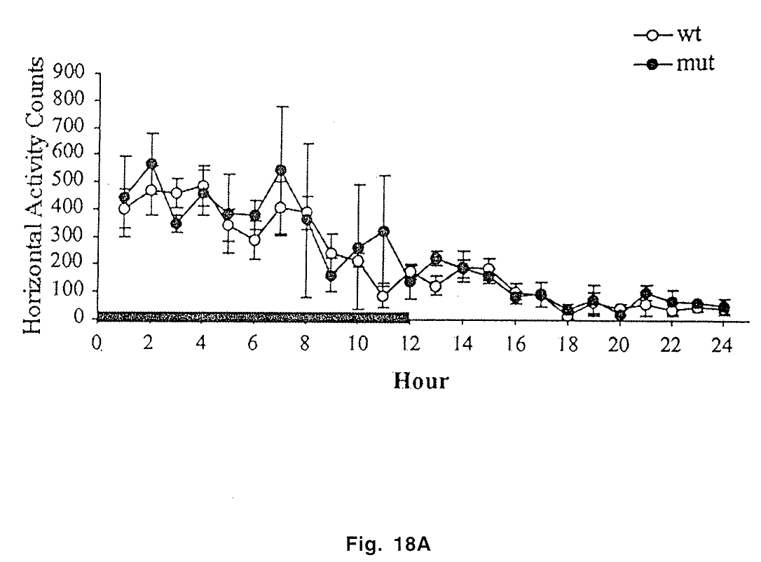

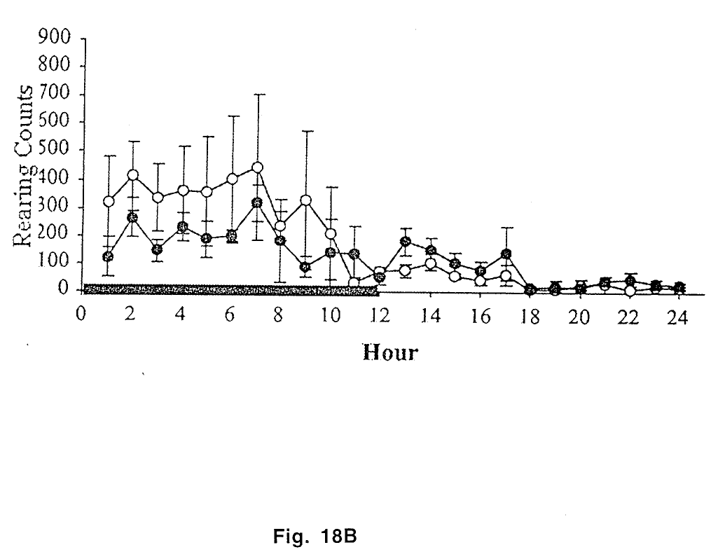

To determine whether the metabolic differences detected in the CRFR2-mutant mice were due to possible differences in basal activity levels, mice were examined for 24-hour locomotor activity. No differences were detected between genotypes for activity levels during either the light or dark cycles as measured by horizontal activity counts in a computerized activity chamger following a 3-hour habituation period (FIG. 5A). Although it did not reach significance, we noted a possible trend in decreased rearing counts in the mutant mice (FIG. 18B). Because decreased rearing behavior is associated with increased anxiety-like behaviors, these results are supportive of the previously reported anxiety-like phenotype of these mice.

The present results demonstrate that CRFR2 is an important component of energy balance regulation. Following homeostatic stressors, such as high-fat diet (Tannenbaum et al., 1997) or repeated cold exposure, CRFR2-mutant mice respond by preferentially depleting their fat stores. Their decreased feed efficiency, compared with wild-type littermates during these challenges, also illustrates the involvement this receptor has in preserving physiological balance. On a high-fat diet, the mutant mice consumed substantially more food while maintaining the same body weight as their wild-type littermates but had lower cholesterol and remained leaner. The percentage of body fat was lower in mutant mice than wild-type mice following the high-fat diet and cold stress, suggesting a possible increase in sensitivity of the sympathetic nervous system in the mutant mice. The increased UCP1 detected in BAT from mutant mice supports a possible increase in sympathetic tone in these mice. Furthermore, increased BAT activity may then compete for serum triglycerides, depleting WAT and BAT stores and decreasing adipose cell size (88). Although it is possible that BAT thermogenesis may be affected in the mutant mice, no differences between genotypes were detected for basal or cold-stressed rectal body temperatures. CRFR2-mutant mice have elevated CRF levels in the central nucleus of the amygdala and UCN1 levels in the edinger westphal nucleus (73). CRF neurons in the central nucleus of the amygdala project to and increase the firing rate of neurons in the locue coeruleus (89) and dorsal raphe (90, 91). Additionally, icv infusion of UCN1 or CRF increases whole-body oxygen consumption and colonic temperature (92, 93) and direct infusion of UCN1 into the paraventricular nucleus of the hypothalamus decreases the repiroatory quotient (94) and increases BAT UCP1 levels (77). These data support the hypothesis that in the absence of CRFR2, unimpeded CRFR1 activity in the mice could be causing increased sympathetic stimulation.

Along with maintenance of body compositon, homeostasis also involves a tight regulation of circulating and stored glucose. Under basal conditions, CRFR2-mutant and wild-type mice have similar glucose and insulin levels. Following a glucose or insulin challenge, however, the mutant nmice showed a lower maximal rise in glucose levels than the wild-type mice, suggesting that the mutant mice may be more sensitive to changes in plasma glucose and more insulin sensitive. While on a high-fat diet, glucose levels in the mutant mice were unaltered, whereas wild-type mice showed a rise in plasma glucose and insulin, indicative of rising insulin resistance that may correspond to increasing body fat in the control mice. Although insulin levels do rise in the mutant mice on the high-fat diet, this increase is significantly lower than that seen in the wild-type mice. Type 2 diabetes and insulin resistance are highly associated with obesity. Although the mechanism for this association is unclear, increasing evidence suggests that increased fat accumulation in the muscle may play a role. These results illustrate a possible role for CRFR2 in insulin sensitivity.

To compare basal activity levels, CRFR2-mutant and wildtype mice were monitored over 24 hours for horizontal and vertical locomotor activity. Results revealed no significant differences between CRFR2-mutant and wild-type mice activity during either the light or dark cycle. The effect of genotype on rearing behavior neared significance because of lightly lower rearing counts in CRFR2-mutant mice relative to controls. This is consistent with greater anxiety-like behavior characteristic and previously reported of these mutant mice (70). These results support the hypothesis that the differences in body composition, food intake and plasma lipids detected in the CRFR2-mutant mice likely are not due to differences in basal activity levels.

Our results support a model in which CRFR1 and CRFR2 play important roles in regulation of organismal responses to stress and perturbations of homeostasis. This model suggests that following such a challenge, CRFR1 stimulates the sympathetic nervous system thereby increasing sympathetic outflow to maintain physiologic equilibrium in the organism under acute perturbations for energy mobilization and redistribution and amy also function in allostasis under more chronic insults. CRFR2, however, appears to function as an inhibitory or modulatory receptor to dampen these actions of CRFR1. In the absence of CRFR2, CRFR1-mediated activity goes unimpeded, as seen in the CRFR2-mutant mice. CRFR2-deficient mice under basal conditions do not display significant differences in food intake or body composition from wild-type littermates, but rather these changes are seen following an insult to their homeostasis, thus supporting the hypothesis that CRFR2 normally functions in such a way as to harness the stimulatory actions of CRFR1. this hypothesis is further supported by the feed efficiency data presented herein. While on a normal diet, the CRFR2-mutant and wild-type mice have a similar feed efficiency, but following exposure to stressors such as a high-fat diet or cold, the mutant mice become metabolically inefficient, causing calories to be wasted as heat and a depletion of fat stores. These results support a role for CRFR2 in the preservation of homeostasis.

### EXAMPLE 27

Increased Depression-Like Behaviors in CRFR2-Deficient Mice: Sexually Dichotomous Responses

CRFR2-deficient and wild-type mice were housed under controlled conditions of 12-hour light/dark (lights on a 6:00 A.M.) with free access to food and water. All procedures were approved by the Salk Institute IACUC.

Depression-Like Behaviors in Modified Forced Swim Test

In order to measure the depression-like behaviors of mice deficient for CRFR2, littermate mice and female CRFR2-mutant and wild-type mice (n=12) were tested in a modified version of the Porsolt Forced Swim Test. The test was modified by increasing the depth of the water in the cylinder to 15 cm above the bottom of the cylinder (98, 112). All animals were placed in the cylinder for a pre-swim for 5 min on day 1 and then monitored during a 5 min test 24 hours following the pre-swim. The time spent swimming, climbing, and immobile were determined by an investigator blind to genotype and treatment. Immobility was defined as time spent still or only using righting movements to remain afloat. Swimming was defined as any movement horizontal in nature that involved at least two limbs. Climbing was defined as any vertical movement in which the bottom of the front paws touche4d the sides of the cylinder.

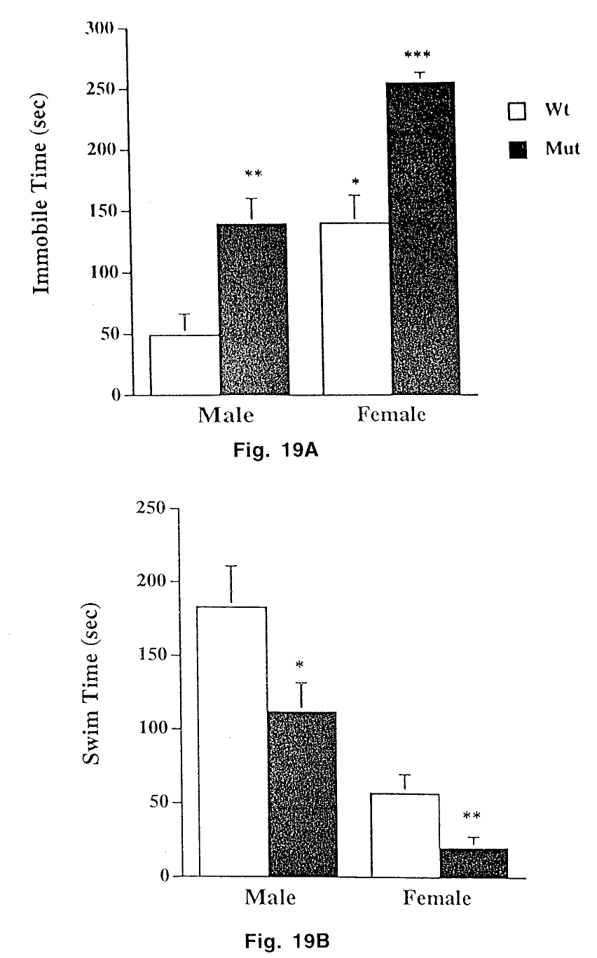

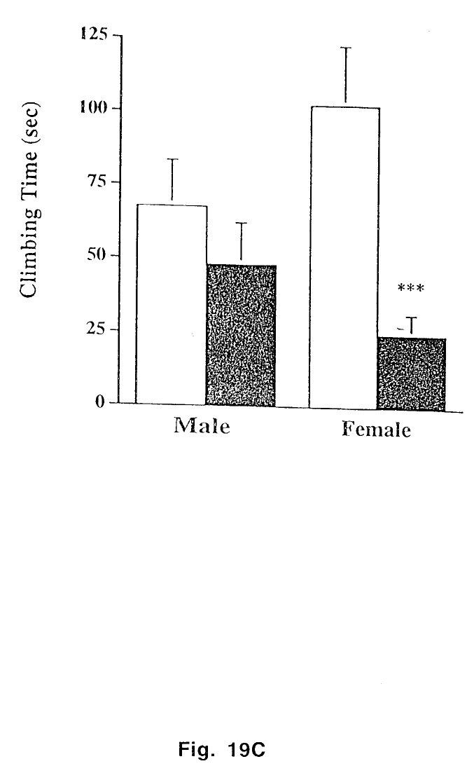

In order to examine depression-like behaviors in mice deficient for CRFR2 compared to wild-type littermates, male and female mice were tested in the Forced Swim Test. The tests revealed a significant increased in immobility time in both male and female CRFR2-mutant mice compared to their wild-type littermates (FIG. 19A). Wild-type and mutant female mice showed significantly more immobile time than their corresponding male mice did. Female and male mutant mice showed a significant decrease in time spent swimming during the test compared to their wild type littermates (FIG. 19B). Female mutant mice also showed a significant decreased in time spent climbing during the test compared to their female wild type littermates (FIG. 19C). No difference in climbing was found between male wild type and mutant mice.

Response to CRFR1 Antagonist (Antalarmin) Treatment

Antalarmin was supplied by Dr. George Chrousos (104, 129). The drug was dissolved in DMSO at 9 mg /ml and diluted in 0.9% saline prior to ip injection. Vehicle was the same concentration of DMSO in 0.9% saline.

In order to determine if antagonism of CRFR1 could reverse the increased depression-like behaviors detected in the CRFR2-mutant mice, male and female CRFR2-deficient mice (n=12) were injected with either antalarmin (7.5 mg/kg, ip) or vehicle (100 ml volume, ip) one hour prior to testing. As above, the preswim and test were 5 min each. Scoring was again performed by an investigator blind to treatment. Mice were tested again either 24 or 72 hours following treatment in another 5 min test. Behaviors were scored as described in Example 27. Results for all tests were averaged and statistics done using Statview (SAS Institute).

In order to determine a possible cause of the increased depression-like behaviors in the CRFR2-deficient mice, a CRFR1 small molecule antagonist, antalarmin, was administered to CRFR²-mutant mice prior to testing in the forced swim test. Previous reports have demonstrated a decrease in immobile time in the forced swim test following antalarmin treatment (103) as well as decreases in depression in humans in response to other CRFR1 non-peptide antagonists (131).

Females

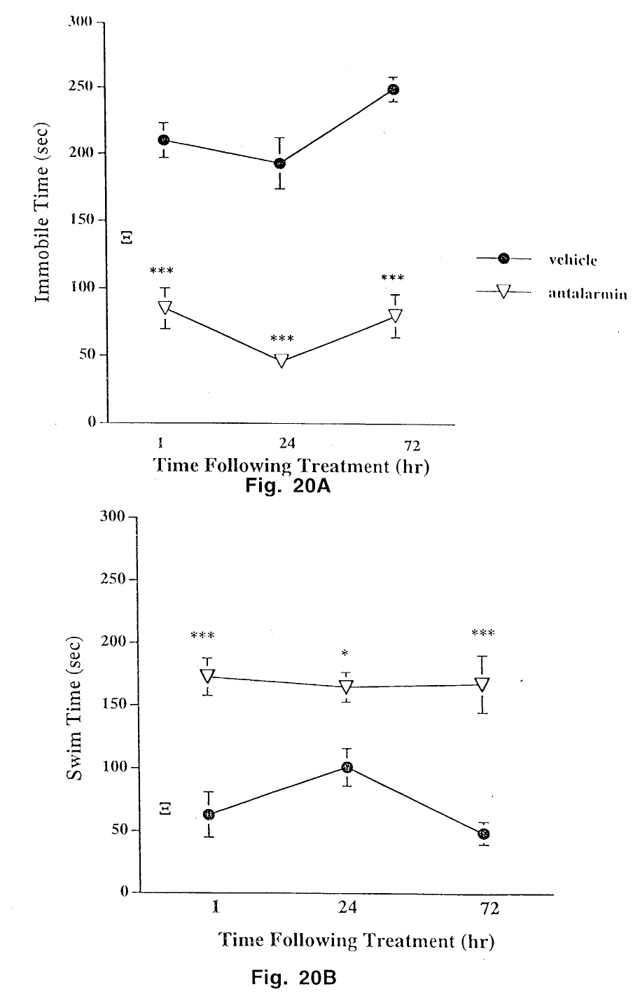

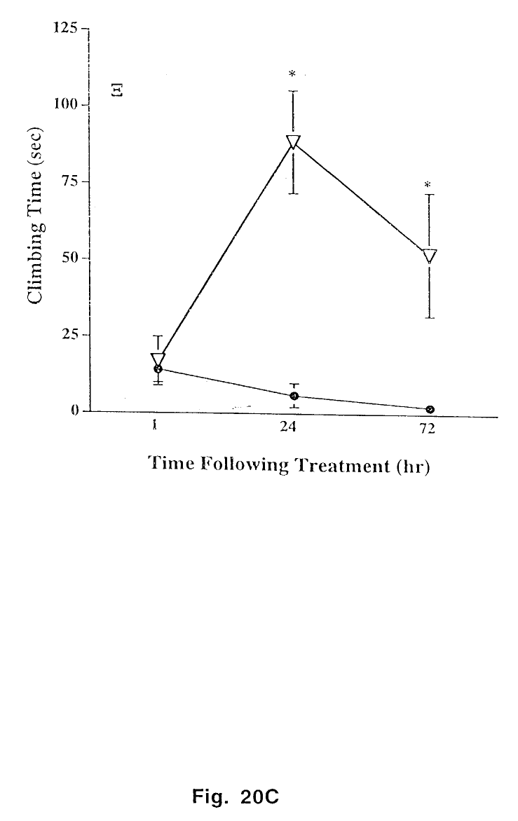

Results revealed a significant decrease in immobile (FIG. 20A) and swim time (FIG. 20B) and an increase in climbing time (FIG. 20C) in female CRFR2-deficient mice pre-treated with antalarmin. The decrease in immobility was detectable 1 hour following treatment in female mutant mice compared to vehicle treated mutant females and remained significantly decreased 24 and 72 hours following treatment (FIG. 20A). Analarmin pretreatment of CRFR2-mutant female mice decreased immobile time by 1 hour to levels slightly lower than untreated wild type female immobile time (levels from FIG. 19A). The increase in swimming was detectable 1 hour following treatment in female mutant mice compared to vehicle treated mutant females and remained significantly increased 24 and 72 hours following treatment (FIG. 20B). A significant increase in time spent climbing was detected 24 hours following antalarmin administration and was still detectable 72 hours following treatment (FIG. 20C). No difference in climbing time was detected 1 hour following antalarmin treatment. Time spent climbing for CRFR2-deficient females 24 hour following antalarmin treatment was similar to that shown for untreated wild type females (levels from FIG. 19C).

Males

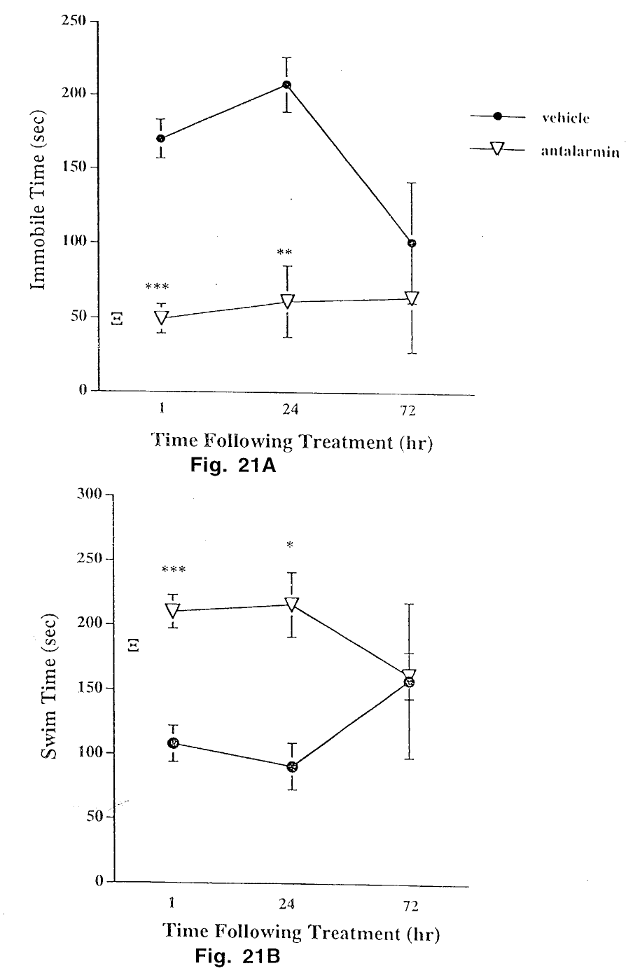

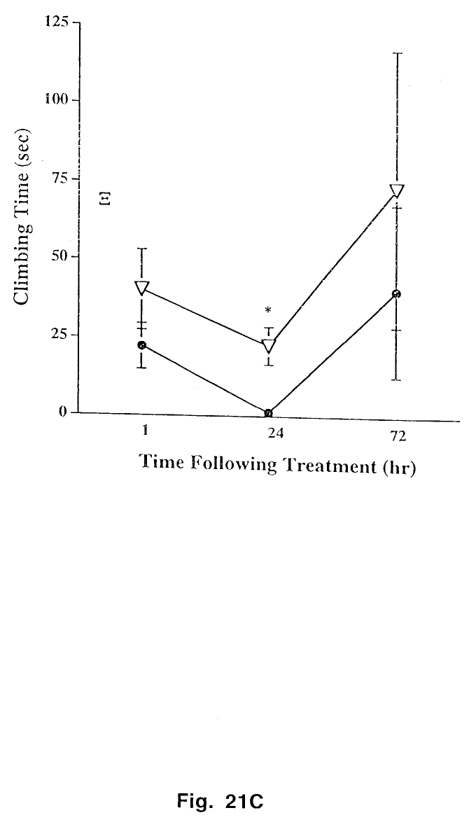

Results revealed a significant decrease in immobile time in male CRFR2-deficient mice 1 and 24 hours following antalarmin treatment compared to vehicle treated male mutant mice (FIG. 21A). The level of immobility detected in the antalarmin treated mutant male mice was similar to that found in untreated wild type male mice (levels from FIG. 19A). Results revealed a significant increase in swim time in male CRFR2-deficient mice 1 and 24 hours following antalarmin treatment compared to vehicle treated male mutant mice (FIG. 21B). The increased level of swimming detected in the antalarmin treated mutant male mice was similar to that found in untreated wild-type male mice (levels from FIG. 19B). A significant increase in time spent climbing was detected in male mutant mice 24 hours following treatment with antalarmin (FIG. 21C). This effect was not significantly different from vehicle treated males 1 or 72 hours following treatment.

CRFR2-deficient mice provide a good model to examine the effects of prolonged stress sensitivity and increased anxiety on the development of depression. Normal mice are typically not useful models for the study of depression as the development of most depressions likely requires a genetic vulnerability (116).

In these studies, male and female CRFR2-deficient mice showed increased depression-like behaviors in the forced swim test. Female mutant mice demonstrated both increased immobility as well as decreased climbing time compared to their wild type female littermates. Male mutant mice also showed a significant increase in immobile time, but no difference was detected in time spent climbing compared to wild-type male mice. These distinct differences in depression behaviors suggest potential roles for CRF receptors in the development and presentation of depression. Treatment with the CRFR1 small molecule antagonist, antalarmin, decreased immobile time and increased climbing time in both CRFR2-deficient male and female mice. These results support the prevailing hypothesis that increased CRFR1 activity results in increased susceptibility for the development of depression, as previous studies nave demonstrated an involvement of unimpeded CRFR1 activity or increased production of CRF with the development of anxiety-like or depression-like behaviors in rodents and humans (122, 93, 107, 131, 103). We have previously reported increased CRF levels in the central nucleus of the amygdala (cAmyg) and increased UCNI levels in the edinger westphal nucleus in CRFR2-deficient mice (95). Increased ligand levels acting at CRFR1 may be influencing neurotransmitter release from brain regions important for normal responses to stress and homeostatic maintenance.

Dysregulation of this pathway may then lead to a proclivity for developing depression-like behaviors as seen in the CRFR2-mutant mice.

The time course and sexually dichotomous response of antalarmin treatment detected in the CRFR2-mutant mice may provide clues as to the neurotransmitters involved in the onset of depression. The modified forced swim test for depression has been demonstrated to be a good model of depression in rodents and is responsive to various antidepressant treatment (102, 112, 98). In this test, antidepressant treatments decrease immobile time, either by increasing swim time and/or increasing climbing time. Studies have categorized these effects as specific to different neurotransmitter pathways, such that catecholaminergic agents may decrease immobility by increasing climbing, while serotonergic agents may decrease immobile time by increasing swimming (112). Based on these studies, results in the CRFR2-deficient mice may indicate that the response to antalarmin on decreasing immobility in both females and males may be attributable to effects on these neurotransmitter pathways. The delayed response detected in male and female mutant mice following antalarmin treatment may also suggest that antagonism of CRFR1 prior to repeated forced swim exposure decreases the onset of depression-like behaviors.

Electrophysiological, biochemical, and anatomical localization studies have shown direct input and potent activation of CRF fibers in the dorsal raphe nucleus (DR) (119, 109, 118, 127). CRF has been shown to affect 5HT release to both the striatum and lateral septum as well as to directly alter DR neuronal activity, where low doses were shown to inhibit 5HT release, and high doses were shown to either increase or have no effect (127). These results may be attributable to the heterogeneity of the DR or to the specific CRF receptor being activated (127, 105). Both CRFR1 and CRFR2 have been detected in the DR and may have opposing roles for 5HT release. As CRF has a 10-fold higher affinity for CRFR1 than CRFR2, low doses of CRF in the DR may preferentially activate CRFR1, where higher doses could potentially stimulate neurons expressing both receptors. Thus, one may hypothesize then that activation of CRFR1 inhibits 5HT release while activation of CRFR2 may augment its release (127, 105). Certainly, a growing body of evidence now supports this hypothesis and has demonstrated CRF receptor specific effects on 5HT fibers. The CRFR1 antagonist, antalarmin, has previously been shown to block the inhibitory effects of cRF by increasing 5HT release (109). Further, CRFR1-deficient mice demonstrate enhanced hippocampal 5HT neurotransmission (117). These results support those previously shown where antagonism of CRFR1 decreases depression-like behaviors in rodents (103) as well as in humans (131). Our results add to this hypothesis by suggesting that in the absence of CRFR2, CRFR1 tone predominates, and may affect 5HT release and increase susceptibility to the development of depression-like behaviors.

In addition to 5HT involvement, catecholamines have also been associated with the development of depression (reviewed in 116). CRF interactions with dopamine (DA) and norepinephrine (NE) neurotransmission have been demonstrated and may involved direct or indirect actions on cell bodies in the locus coeruleus (LC) or ventral tegmental area (VTA) (113, 123, 99, 111, 108). CRFR1 has been detected in both the VTA (121) and in the LC (126, 128). Antagonism of CRFR1 has been demonstrated to inhibit discharge from the LC (99, 100). Results from our studies show a response of CRFR2-deficient male and female mice to antalarmin treatment with respect to climbing behavior in the forced swim test. Increased climbing behavior has been associated with changes in catecholaminergic neurotransmission (98), and therefore may indicate a possible involvement of the CRF system with catecholaminergic pathways.

Depression is twice as common in females as it is in males. The basis for this increased susceptibility is not known, but may involve sex differences in hormonal or stress response pathways (130) or sexually dimorphic brain regions important in the neurobiology of depression. In deciphering the mechanisms involved in these sex differences, we examined both males and females for depression-like behaviors. Results showed a significant increase in immobile time in wild-type and CRFR2-mutant females compared to males of the same genotype. Pre-treatment with antalarmin of both males and females demonstrated a rapid decreased in immobile time following treatment. However, the response was longer lasting in females. Female CRFR2-deficient mice also displayed a more pronounced increase in climbing behavior compared to mutant males, providing further evidence of possible CRF-stress pathway involvement in the increased susceptibility of females for depression. These results provide support for further examination of stress responsiveness as an indicator of susceptibility to depression, especially in females.

In summary, our results have identified a significant increase in depression-like behaviors in CRFR2-deficient mice. Further, we have demonstrated sexually dichotomous responses to antalarmin treatment in depression-like behaviors between CRFR2-mutant males and females, establishing possible interactions with CRF in the development and increased sensitivity of females for depression. Elevated CRF and UCNI levels in these mice may be contributing to the increased depression-like behavioral responses detected. These results also suggest that mice may be good animal models for examining sex differences in depression. Further studies examining the involvement of CRF family members as well as distinct neurotransmitter systems will provide necessary information as to the genetic and neurobiological basis relating stress and depression and the increased risk for females.

The following references were cited herein:

1. Vale, et al., Science 213, 1394-1397 (1981).

2. Vaughan, J. , et al., Nature 378, 287-292 (1995).

3. Perrin et al. Proc Natl Acad Sci USA 92, 2969-2973 (1995).

4. Lovenberg, T. W. et al. [published erratum appears in Proc Natl Acad Sci USA Jun. 6, 1995;92(12):5759]. Proc Natl Acad Sci USA 92, 836-840 (1995).

5. Kishimoto, , et al., [published erratum appears in Proc Natl Acad Sci USA Apr. 25, 1995;92(9):4074]. Proc Natl Acad Sci USA 92, 1108-1112 (1995).

6. Stenzel, P. et al. Mol Endocrinol 9, 637-645 (1995).

7. Chalmers, , et al., J Neurosci 15, 6340-6350 (1995).

8. Smith, G. W. et al. Neuron 20, 1093-1102 (1998).

9. Timpl, P. et al. Nat Genet 19, 162-166 (1998).

10. Webster, E. L. et al. Endocrinology 137, 5747-5750 (1996).

11. Bornstein, S. R. et al. Endocrinology 139, 1546-1555 (1998).

12. Deak, T. et al. Endocrinology 140, 79-86 (1999).

13. Spina, M. et al. Science 273, 1561-1564 (1996).

14. Schilling, et al., Br j Pharmacol 125, 1164-1171 (1998).

15. Bale, et al., Endocrinology 136, 27-32

16. Hogg, S. Pharmacol Biochem Behav 54, 21-30 (1996).

17. Rodgers, R. J. Animal models of ‘anxiety’: where next? Behav Pharmacol 8, 477-496; discussion 497-504 (1997).

18. Belzung, C. & Le Pape, G. Physiol Behav 56, 623-628 (1994).

19. Palmiter, et al., Recent Prog Horm Res 53, 163-199 (1998).

20. Heilig, et al., Regul Pept 41, 61-69 (1992).

21. Liang, K. C. et al. J Neurosci 12, 2313-2320 (1992).

22. King, F. A. & Meyer, P. M. Science 128, 655-656 (1958).

23. Melia, et al., Physiol Behav 49, 603-611 (1991).

24. Lee, et al., J Neurosci 17, 6424-6433 (1997).

25. Allen, et al., Neuroendocrinology 19, 115-125 (1975).

26. Beaulieu, et al., Neuroendocrinology 44, 247-254 (1986).

27. Beaulieu, et al., Neuroendocrinology 45, 37-46 (1987).

28. Marcilhac, et al., Exp Physiol 81, 1035-1038 (1996).

29. Moreau, et al., Neuroreport 8, 1697-1701 (1997).

30. K. Sakai, et al., Brain Res 119, 21-41 (1977).

31. Butler, et al., J Neurosci 10, 176-83 (1990); J. M. Weiss, et al., Brain Res Bull 35, 561-72 (1994).

32. Udelsman, et al., Nature 319, 147-150 (1986).

33. Andreis, et al., Endocrinology 128, 1198-1200 (1991).

34. Andreis, et al., Endocrinology 131, 69-72 (1992).

35. Heinrichs, et al., Regul Pept 71, 15-21 (1997).

36. Liebsch, et al., J Psychiatr Res 33, 153-163 (1999).

37. Negri et al., Peptides 6: 53-57.

38. Heinrichs et al., Peptides 13: 879-884 (1992).

39. Spina et al., Science 273: 1561-1564 (1996).

40. Contarino et al., Endocrinology 141: 2698-2702 (2000).

41. Bradbury et al., Endocrinology 141: 2715-2724 (2000).

42. Cullen et al., Endocrinology 142: 992-999 (2001).

43. Britton et al., Brain Res. 369: 303-306 (1986).

44. Liang et al., J. Neurosci. 12: 2303-2312 (1992).

45. Stenzel-Poore et al., J. Neurosc.i 14: 2579-2584 (1994).

46. Buwalda et al., Psychoneuroendocrinology 22: 297-309 (1997).

47. Brown et al., Regul. Peptides 4: 107-114 (1982).

48. Aguilera et al., J. Biol. Chem. 258: 8039-8045 (1983).

49. Rivier et al., Endocrinology 115: 882-886 (1984).

50. Pich et al., Psychoneuroendocrinology 18: 495-507 (1993).

51. Menzaghi et al., J. Pharmacol. Exp. Ther. 269: 564-572 (1994).

52. Vaughan et al., Nature 378: 287-292 (1995).

53. Chen et al., Proc. Natl. Acad. Sci. USA 90: 8967-8971 (1993).

54. Perrin et al., Proc. Natl. Acad. Sci. USA 92: 2969-2973 (1995).

55. Lovenberg et al., Proc. Natl. Acad. Sci. USA 92: 836-840 (1995).

56. Kishimoto et al., Proc. Natl. Acad. Sci. USA 92: 1108-1112 (1995).

57. Preil et al., Endocrinology 142: 4946-4955 (2001).

58. Smagin et al., Neurosci. Lett. 220: 167-170 (1996).

59. Rodriguez et al., J. Pharmacol Exp. Ther. 276: 56-64 (1996).

60. Lundkvist et al., Eur. J. Pharmacol. 309: 195-200 (1996).

61. Baram et al., Brain Res. 770: 89-95 (1997).

62. Griebel et al., Psychopharmacology (Berl.) 138: 55-66 (1998).

63. Timpl et al., Nat. Genet. 19: 162-166 (1998).

64. Smith et al., Neuron 20: 1093-1102 (1998).

65. Contarino et al., Brain Res. 835: 1-9 (1999).

66. Liebsch et al., J. Psychiatr. Res. 33: 153-163 (1999).

67. Heinrichs et al., Regul. Pept. 71: 15-21 (1997).

68. Martinez et al., Am. J. Physiol. 274: G965-G970 (1998).

69. Smagin et al., Neuroreport 9: 1601-1606 (1998).

70. Bale et al., Nat. Genet. 24: 410-414 (2000).

71. Coste et al., Nat. Genet. 24: 403-409 (2000).

72. Kishimoto et al., Nat. Genet. 24: 415-419 (2000).

73. Bale et al., J. Neurosci. 22: 193-199 (2002).

74. Brown et al., Endocrinology 111: 928-931 (1982).

75. Brown et al., Life Sci. 30: 207-210 (1982).

76. Rivest et al., Am. J. Physiol. 257: R1417-R1422 (1989).

77. Kotz et al., Am. J. Physiol. Regul. Integr. Comp. PhysioL. 282: R546-R551 (2002).

78. Egawa et al., Am. J. Physio. 259: R799-R806 (1990).

79. Egawa et al., Neuroscience 34: 771-775 (1990).

80. Dunn et al., Pharmacol. Biochem. Behav. 27: 685-691 (1987).

81. Andreis et al., Endocrinology 128: 1198-1200 (1991).\

82. Nijsen et al., Neuropsychopharmacolgy 22: 388-399 (2000).

83. Hashimoto et al., Endocr. J. 48: 1-9 (2001).

84. Makino et al., Neuroendocrinology 70: 160-167 (1999).

85. Tannenbaum et al., Am. J. Physiol. 273: E1168-E1177 (1997).

86. Cummings et al., Nature 382: 622-626 (1996).

87. Valentino, Psychopharmacol. Bull. 25: 306-311 (1989).

88. Price et al., Neuropsychopharmacology 18: 492-502 (1998).

89. Kirby et al., Neuropsychopharmacology 22: 148-162 (2000).

90. Dagnault et al., Am. J. Physiol. 272: R311-R317 (1997).

91. De Fanti et al., Brain Res. 930: 37-41 (2002).

92. Currie et al., Brain Res. 916: 222-228 (2001).

93. Arborelius et al., J. Endocrinol. 160: 1-12 (1999).

94. Bale et al., J. Neurosci. 22: 193-199 (2002).

95. Bale et al., Nat. Genet. 24: 410-414 (2000).

96. Chalmers et al., J. Neurosci. 15: 6340-6350 (1995).

97. Coste et al., Nat. Genet. 24: 403-409 (2000).

98. Cryan et al., Trends Pharmacol. Sci. 23: 238-245 (2002).

99. Curtis et al., Brain Res. Bull. 35: 581-587 (1994).

100. Curtis et al., J. Pharmacol. Exp. Ther. 281: 163-172 (1997).

101. Curtis et al., J. Pharmacol. Exp. Ther. 268: 359-365 (1994).

102. Detke et al., Exp. Clin. Psychopharmacol. 5: 107-112 (1997).

103. Griebel et al., J. Pharmacol. Exp. Ther. 301: 333-345 (2002).

104. Habib et al., Proc. Natl. Acad. Sci. USA 97: 6079-6084 (2000).

105. Hammack et al., J. Neurosci. 22: 1020-1026 (2002).

106. Heim et al., Biol. Psychiatry 46: 1509-1522 (1999).

107. Holsboer, J. Pshchiatr. Res. 33: 181-214 (1999).

108. Kawahara et al., Eur. J. Pharmacol. 387: 279-286 (2000).

109. Kirby et al., Neuropsychopharmacology 22: 148-162 (2000).

110. Kishimoto et al., Nat Genet. 24: 415-419 (2000).

111. Lechner et al., Brain Res. 756: 114-124 (1997).

112. Lucki, Behav. Pharmacol. 8: 523-532 (1997).

113. Melia et al., Proc. Natl. Acad. Sci. USA 88:8382-8386 (1991).

114. Nemeroff, Pharmacopsychiatry 21: 76-82 (1988).

115. Nemeroff, Neuropsychopharmacology 6: 69-75 (1992).

116. Nestler et al., Neuron 34: 13-25 (2002).

117. Penalva et al., Neuroscience 109: 253-266 (2002).

118. Price et al., J. Neurosci. 21: 2833-2841 (2001).

119. Price et al., Neuropsychopharmacology 18: 492-502 (1998).]

120. Reul et al., Curr. Opin. Pharmacol. 2: 23-33 (2002).

121. Sauvage et al., Neuroscience 104: 643-652 (2001).

122. Stenzel-Poore et al., J. Neurosci 14: 25799-2584 (1994).

123. Swiergiel et al., Brain Res. 587: 263-268 (1992).

124. Valdez et al., Brain Res. 943: 142-150 (2002).

125. Vale et al., Science 213: 1394-1397 (1981).

126. Valentino, Psychopharmacol. Bull. 25: 306-311 (1989).

127. Valentino et al., J. Comp. Neurol. 4335: 450-463 (2001).

128. Valentino et al., Neuroscience 48: 689-705 (1992).

129. Webster et al., Endocrinology 137: 5747-5750 (1996).

130. Young, J. Gend. Specif. Med. 1: 21-27 (1998).

131. Zobel et al., J. Psychiatr Res. 34: 171-181 (2000).

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A non-natural transgenic mouse with a disruption in at least one allele of the corticotropin releasing factor receptor 2 (CRFR2) such that said mouse does not express corticotropin releasing factor receptor 2 protein from said allele.

2. The transgenic mouse of claim 1, wherein the DNA sequences for exons 10, 11, and 12 of said corticotropin releasing factor receptor 2 allele have been deleted.

3. The transgenic mouse of claim 2, wherein said DNA sequences have been replaced with a neomycin resistance gene cassette.

4. The transgenic mouse of claim 3, wherein said mouse is heterozygous for said replacement.

5. The transgenic mouse of claim 3, wherein said mouse is homozygous for said replacement.

6. The progeny of a mating between a mouse of claim 3 and a mouse of another strain.

7. A method of screening a compound for anxiety modulating activity, comprising the steps of:
a) administering said compound to the transgenic mouse of claim 5;
b) testing said mouse for anxiety-related behavior; and,
c) comparing anxiety-like behavior of said mouse with anxiety-like behavior in a second transgenic mouse of claim 5 to which said compound was not administered.

8. The method of claim 7, wherein said mice are tested for anxiety in an elevated plus maze.

9. A method of screening a compound for depression-modulating activity, comprising the steps of:
a). administering said compound to the transgenic mouse of claim 5;
b). testing said mouse for depression-like behavior; and,
c). comparing depression-like behavior of said mouse with depression-like behavior in a second transgenic mouse of claim 5 to which said compound was not administered.

10. The method of claim 9, wherein said mice are tested for depression-like behavior in a forced swim test.

11. The method of claim 9, wherein said compound is a CRFR1 antagonist.

12. The method of claim 9, wherein said comparing depression-like behavior is between a male and a female transgenic mouse.

13. A method of treating a pathological condition related to depression, comprising the step of administering an effective dose of the compound of claim 9 to an individual in need of such treatment.

14. A pharmacological composition comprising the compound of claim 9.

15. A method of screening for compounds which control blood pressure, comprising the steps of:
a). administering a compound to the transgenic mouse of claim 5;
b). testing said transgenic mouse for alterations in blood pressure; and,
c). comparing alterations of blood pressure in said transgenic mouse with alterations of blood pressure in a second mouse, wherein said second mouse is selected from the group consisting of a transgenic mouse of claim 5 to which said compound was not administered and a wild type mouse to which said compound was also administered.

16. A method of screening for compounds which affect angiogenesis, comprising the steps of:
a). administering a compound to the transgenic mouse of claim 5;
b). assaying said transgenic mouse for alterations in angiogenesis; and,
c). comparing alterations of angiogenesis in said transgenic mouse with alterations of angiogenesis in mice selected from the group consisting of transgenic mice of claim 5 to which said compound was not administered and wild type mice to which said compound was administered.

17. A method of screening a compound for effects on the response of the hypothalamic-pituitary-adrenal axis to stress, comprising the steps of:
a). administering said compound to the transgenic mouse of claim 5;
b). placing said mouse in a stress-inducing situation,
c). monitoring plasma levels of corticosterone and adrenocorticotropic hormone in said mouse; and,
d). comparing said levels to those in a transgenic mouse of claim 5 not placed in said stress-inducing situation.

18. The method of claim 17, wherein said stress-inducing situation is physical restraint-stress.

19. A method of determining the effects of CRFR2 on a second protein, comprising the steps of
a). administering an agonist that affects the second protein to the transgenic mouse of claim 5;
b) performing an assay of the second protein, wherein said assay is selected from the group consisting of assays of protein expression and assays of protein activity; and,
c). comparing assay results on said transgenic mouse with those obtained from a wild type mouse administered the same agonist.

20. The method of claim 19, wherein said second protein is selected from the group consisting of corticotropin releasing factor, corticotropin releasing factor receptor 1, urocortin, corticotropin receptors and urocortin receptors.

21. A method of stimulating increased angiogenesis in a target tissue comprising the step of administering a CRFR2 antagonist to said target tissue.

22. The method of claim 21, wherein said CRFR2 antagonist is an antisense nucleotide directed against CRFR2.

23. The method of claim 21, wherein said target tissue is selected from the group consisting of heart, brain, pituitary, gonad, kidney, adipose, and gastrointestinal tract tissues.

24. The method of claim 21 wherein said angiogenesis is increased in an individual having a pathophysiological condition selected from the group consisting of infarction, stroke, and injury.

25. A method of inhibiting angiogenesis in a target tissue comprising the step of administering a CRFR2 agonist to said target tissue.

26. The method of claim 25 wherein said CRFR2 agonist is selected from the group consisting of urocortin and CRF.

27. The method of claim 25, wherein said tissue is selected from the group consisting of heart, brain, pituita gonad, kidney, adipose, and gastrointestinal tract tissues.

28. The method of claim 25 wherein said angiogenesis is inhibited in an individual having a pathophysiological condition selected from the group consisting of cancer and diabetic retinopathy.

29. A method of stimulating hair growth comprising the step:
contacting urocortin with a region of skin on which hair growth is desired.

30. The method of claim 29, wherein said urocortin is implanted under the skin.

31. The method of claim 29, wherein bFGF is administered to said skin before urocortin, after urocortin or simultaneously with urocortin.

32. The method of claim 29, wherein urocortin is contained in a composition with bFGF.

33. A method of screening a compound for effects on a response to stress on homeostasis, comprising the steps of:
a). administering said compound to a first wild-type mouse;
b). placing said first wild-type mouse, a second wild-type mouse, and the transgenic mouse of claim 5 in a stress-inducing situation,
c). monitoring said response to stress in said first wild-type and said transgenic mouse; and,
d). comparing the response to stress in the wild-type mouse to the response in the transgenic mouse to the response of a second wild-type mouse to which said compound was not administered.

34. The method of claim 33, wherein said stress-inducing situation is selected from the group consisting of a high-fat diet, repeated cold stress, glucose challenge, and insulin challenge.

35. The method of claim 33, wherein the monitoring of said response is selected from the group consisting of analysis of body composition, plasma lipid analysis, tissue histology, Western blot analysis, and analysis of locomotor activity.

36. The method of claim 34, wherein the response of the first wild type mouse and the transgenic mouse to said high-fat diet is selected from the group consisting of lower body fat but higher food intake, no elevation in plasma glucose levels, and a slight rise in plasma insulin levels compared with said second wild type mouse.

37. The method of claim 34, wherein the response of the first wild type mouse and the transgenic mouse to said repeated cold stress is selected from the group consisting of weight loss, lower feed efficiency, and lower body fat compared to the second wild type mouse.

38. The method of claim 34, wherein the response of the first wild type mouse and the transgenic mouse to said glucose challenge comprises a lower peak plasma glucose level compared to the second wild type mouse.

39. The method of claim 34, wherein the response of the first wild type mouse and the transgenic mouse to said insulin challenge is selected from the group consisting of a lower peak plasma glucose level, a more rapid decline in plasma glucose levels, and an increase in insulin sensitivity compared to the second wild type mouse.

40. The method of claim 33, wherein said compound is an antagonist of CRFR2 activity.

41. The method of claim 33, wherein said compound is an agonist of CRFR1 activity.

42. A method of treating a pathological condition, comprising the step of administering an effective dose of the compound of claim 33 to an individual in need of such treatment.

42. A pharmacological composition comprising the compound of claim 33.

43. The method of claim 42, wherein said pathological condition is selected from the group consisting of obesity and type 2 diabetes.
